(19) United States
(12) Patent Application Publication
(10) Pub. No.: US 2012/0108451 A1
(43) Pub. Date: May 3, 2012

(54) METHODS AND APPARATUS FOR NANOPARTICLE-ASSISTED NUCLEIC ACID HYBRIDIZATION AND MICROARRAY ANALYSIS

(75) Inventors: Paul Chi Hang LI, Coquitlam (CA); Lin WANG, Burnaby (CA)

(73) Assignee: SIMON FRASER UNIVERSITY, Burnaby (CA)

(21) Appl. No.: 12/916,455

(22) Filed: Oct. 29, 2010

(51) Int. Cl. C40B 30/04; C40B 60/12; G01N 33/553; G01N 33/543; C40B 50/14; B82Y 15/00

(52) U.S. Cl. 506/9; 436/501; 506/30; 506/39; 977/773; 977/924

(57) ABSTRACT

The invention provides nucleic acid hybridization methods for detecting target nucleic acid sequences wherein complexes comprising nanoparticles non-covalently associated with single-stranded tartlet nucleic acid molecules are incubated with immobilized probe nucleic acid molecules. Because the nanoparticles function as competitors in the hybridization reaction between the target nucleic acid molecules and the probe nucleic acid molecules. The methods provide a high degree of discrimination between a perfectly matched target sequence and a sequence having at least a single-base-pair mismatch, even when the hybridization reaction is performed at room temperature. The invention also provides microarray methods and apparatus which incorporate the nanoparticle-assisted hybridization methods.

### TECHNICAL FIELD

The present invention relates to methods and apparatus for nucleic acid hybridization and microarray analysis.

### BACKGROUND

The ability to detect specific nucleic acid sequences is important for clinical diagnosis, biomedical research, food and drug testing, environmental monitoring and industrial applications. For example, detection of variations in nucleic acid sequences (e.g., single nucleotide polymorphisms (SNPs) in the human genome) is important for studying the genetic basis of inherited diseases and for facilitating individualized medicine.

Nucleic acid hybridization assays have been used for detection of specific nucleic acid sequences. The principle of nucleic acid hybridization has also allowed the development of microarray technology. Microarray technology provides parallel nucleic acid hybridizations for a large number of immobilized oligonucleotides or DNA on a relatively small surface.

However, a central challenge to conventional nucleic acid hybridization assays and microarray technology is achieving a high degree of discrimination between a target sequence that is perfectly matched to a probe and a sequence having a single base-pair mismatch to the probe. In conventional microarray assays, single-base-pair discrimination is typically achieved by adjusting the hybridization conditions (e.g., increasing incubation temperature, or lowering ionic strength, or adding additives such as formamide). In order to reduce or minimize non-specific binding between mismatched target and probe sequences, conventional microarray analysis is typically performed using an incubation temperature in the range of 40 to 65° C. This usually requires the use of temperature control equipment, for example, an incubation oven. Further, conventional microarray analysis is typically performed at elevated temperatures and the incubation requires an extended period of time, for example, from 8 to 20 hours. Additionally, because microarray analysis involves a large set of different probes having different sequences on a single glass slide, it is difficult to find an optimum incubation temperature that would provide good binding specificity for all the target/probe pairs.

Nanoparticles often exhibit properties that differ significantly from those observed in bulk materials. Some of the properties of nanoparticles are their (1) small size (typically 1-100 nm), (2) large surface-to-volume ratio, and (3) unusual target binding properties.

One of the unusual properties about nanoparticles, such as gold nanoparticles, is that single-stranded nucleic acid molecules can non-covalently bind or adsorb to nanoparticles, whereas double-stranded nucleic acid molecules generally do not: Huixiang Li and Lewis Rothberg, 2004, Proceedings of the National Academy of Science, Vol. 101, No. 39, 14036-14039, which is hereby incorporated by reference. Silver nanoparticles have also been shown to behave like gold nanoparticles in terms of non-covalent binding with single-stranded nucleic acid molecules: Chen et al., Analyst, (2010), 135, 1066-1069, which is hereby incorporated by reference. It should be noted that this type of binding between single-stranded nucleic acid molecules and nanoparticles is non-covalent in nature and does not require the formation of covalent bonds.

The different propensities of single-stranded and double-stranded nucleic acid molecules to non-covalently adsorb to nanoparticles arise because single-stranded nucleic acid molecules can uncoil sufficiently to expose their bases, whereas double-stranded nucleic acid molecules have a stable double-helix that presents the negatively charged phosphate backbone. Gold or silver nanoparticles in solution are typically stabilized by adsorbed negatively charged ions (e.g., citrate) whose repulsion prevents the nanoparticles from aggregating. Repulsion between the charged phosphate backbone of double-stranded nucleic acid molecules and the adsorbed citrate ions dominates the electrostatic interaction between the nanoparticles and double-stranded nucleic acid molecules so that double-stranded nucleic acid molecules will not adsorb to the nanoparticles. In contrast, because the single-stranded nucleic acid molecules are sufficiently flexible to partially uncoil the bases, they can be exposed to the nanoparticles. Under these conditions, the negative charge on the phosphate backbone is sufficiently distant so that attractive electrostatic interactions, van der Waals forces and hydrophobic interactions between the bases and the gold nanoparticles are sufficient to cause single-stranded nucleic acid molecules to stick to the nanoparticles. The same mechanism is not operative with double-stranded nucleic acid molecules because the duplex structure does not permit the uncoiling needed to expose the bases: Li and Rothberg, 2004, supra.

Rothberg et al. have developed a method for detection of DNA sequences based on the interaction of single-stranded DNA molecules and gold nanoparticles: Li and Rothberg, supra; Rothberg et al., US Patent Application Publications US 2005/0059042 and US 2006/0166249. The Rothberg method requires a target single-stranded DNA molecule to be first exposed to a probe single-stranded DNA molecule in solution, and then exposed to gold nanoparticles. If the target molecule hybridizes to the probe molecule in solution, the target molecule will not subsequently adsorb to the gold nanoparticles. If the target molecule does not hybridize to the probe molecule in solution, the target molecule will then adsorb to the gold nanoparticles. The differences may be detected by adding salt to the solution, which results in color changes as gold nanoparticles that are not stabilized by single-stranded DNA molecules aggregate under high salt conditions.

In the Rothberg method, the hybridization of target and probe DNA molecules occurs in the absence of gold nanoparticles. In other words, gold nanoparticles are not added before the hybridization reaction, but are added after hybridization to adsorb unhybridized single-stranded DNA molecules. Furthermore, in order to distinguish single-base-pair mismatches, the Rothberg method requires a dehybridization step which appears to require elevated temperatures (e.g., above the melting temperature of mismatched duplexes, but below the melting temperature of perfectly matched duplexes). Furthermore, the Rothberg method does not use immobilized DNA molecules.

Based on the foregoing, it would be desirable to provide nucleic acid hybridization and microarray methods and apparatus that provide a high degree of discrimination between a perfectly matched target sequence and a sequence with a single base-pair mismatch. In particular, it would be desirable to provide methods and apparatus which utilize the unique property of nanoparticles, while avoiding the need for elevated temperatures. For example, it would be desirable to provide such methods and apparatus wherein the incubation step can be performed at ambient temperature without the use of any specialized heating equipment.

### BRIEF DESCRIPTION OF DRAWINGS

In drawings which show non-limiting embodiments of the invention:

FIG. 1 illustrates a nucleic acid hybridization method according to an example embodiment of the invention.

FIG. 1A illustrates a nucleic acid hybridization method according to an example embodiment of the invention, wherein the target nucleic acid molecules are labeled with a detectable label.

FIGS. 2A-C illustrate probe nucleic acid molecules immobilized on substrate surfaces.

FIG. 3 illustrates a nucleic-acid-hybridization-based method to distinguish between two different nucleic acid molecules according to an example embodiment of the invention.

FIG. 4 illustrates an alternative nucleic-acid-hybridization-based method to distinguish between two different nucleic acid molecules according to an example embodiment of the invention.

FIG. 5A illustrates a method for microarray analysis according to an example embodiment of the invention.

FIG. 5B illustrates a method for microarray analysis according, to an example embodiment of the invention, wherein the microarray comprises line arrays.

FIG. 6 illustrates a method for microarray analysis according to an example embodiment of the invention.

FIG. 7 is a schematic view showing the assembly steps for fabricating a microarray device by combining a test chip and first and second channel plates.

FIG. 8A is a schematic view of a first channel plate having a plurality of first microfluidic channels configured in a radial pattern.

FIG. 8B is a schematic view of second channel plate having a plurality of second microfluidic channels configured in a spiral pattern.

FIG. 8C is schematic view showing a first dimensional, centrifugal force (F) used to distribute liquids in the radially configured first microfluidic channels of FIG. 8A.

FIG. 8D is a schematic view showing a second dimensional centrifugal force used to distribute liquids in the spiral second microfluidic channels of FIG. 8B.

FIG. 8E is a schematic view showing the first channel plate sealingly connected to a test chip;

FIG. 8F is a schematic view, showing the second channel plate sealingly connected to the test chip after removal of the first channel plate.

FIG. 8G is a schematic view of a test chip showing, positive test results at select microarray test positions after removal of the second channel plate.

FIG. 9A is a schematic view of a blank test chip.

FIG. 9(B1) is a schematic view of a first channel plate having a plurality of first microfluidic channels configured in a right spiral pattern.

FIG. 9(B2) is a schematic view of a second channel plate having a plurality of second microfluidic channels configured in a left spiral pattern.

FIG. 9C is a schematic view showing the intersecting reagent distribution patterns on the test chip.

FIG. 9D is a schematic view showing positive test results at microarray test positions located at the intersections between the reagent distribution patterns of FIG. 9C.

FIG. 10A is a plan view of a first channel plate having a plurality of closely spaced first microfluidic channels configured in a right spiral pattern. The inset shows selected channels in fluid communication with fluid inlet reservoirs.

FIG. 10B is a plan view of a second channel plate having a plurality of closely spaced second microfluidic channels conFig.d in a left spiral pattern. The inset shows selected channels in fluid communication with fluid inlet reservoirs.

FIG. 10C is a plan view showing the intersecting reagent distribution patterns applied to a test chip. The inset shows selected test positions formed by the intersections of the first and second reagent distribution patterns.

FIG. 11 illustrates a microfluidic microarray method using straight microchannels: (a) creation of a DNA probe line array on aldehyde-modified glass slide via straight microchannels; (h) hybridization of DNA samples in straight channels orthogonal to the straight probe lines printed on the glass slide.

FIG. 12 illustrates an example PDMS microfluidic microarray assembly: (a) a first channel plate used for probe immobilization: (h) a second channel plate used for sample hybridization; (c) a cross-sectional view of the 16 microchannels along line AB in (b).

FIG. 13 illustrates non-covalent association and dissociation between DNA molecules and gold nanoparticles (GNPs). (a) GNP-DNA complexes from the incubation of target DNA with GNP; (b) perfectly matched target DNAs desorbed from GNPs and hybridized to the surface immobilized probes. (c) mismatched DNAs remained bound to GNPs and were washed away.

FIG. 14 shows (a) images of hybridized patches of perfectly matched (PM) and single-base-pair-mismatched (MM) target oligonucleotides in triplicate. Here, the oligonucleotides (oligos) were preincubated with GNPs (5 nm) at different ratios. (b) Discrimination ratios between PM and MM duplexes. The discrimination ratios were calculated by dividing the signal of PM DNAs with that of MM DNAs (the higher ratio, the better discrimination). (c) The fluorescent hybridization signals from the images in (a), and the results at oligo/GNPs=1:1 are expanded and shown in the right inset.

FIG. 15 shows (a) images of hybridized patches of PM and MM (264-bp) PCR products or amplicons in triplicate. Here, the amplicons were preincubated with GNPs (5 nm) at different ratios. (b) Discrimination ratios between PM and MM amplicons. The discrimination ratios were calculated by dividing the signal of PM DNAs with that of MM DNAs.

### DETAILED DESCRIPTION

Throughout the Following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

### Nanoparticle-Assisted Nucleic Acid

One aspect of the invention provides a method for nanoparticle-assisted nucleic acid hybridization analysis. The method has many applications. For example, the method can be used to assist the detection of the presence or absence of a specific target nucleic acid molecule in a sample or test solution.

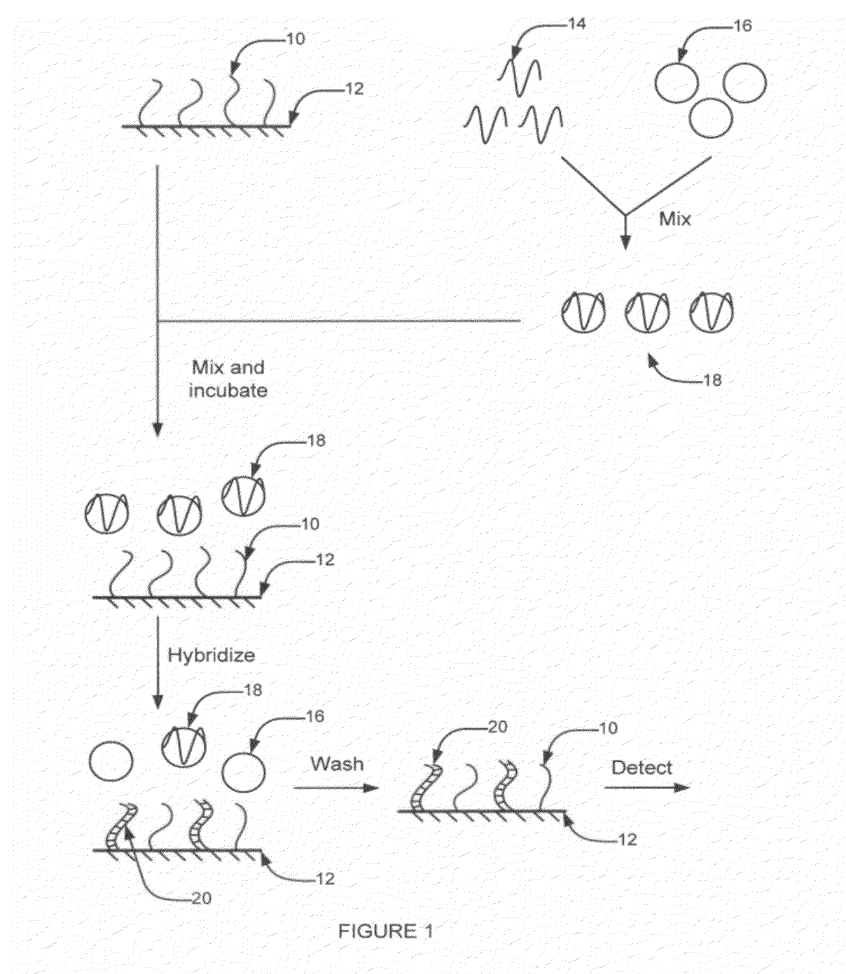

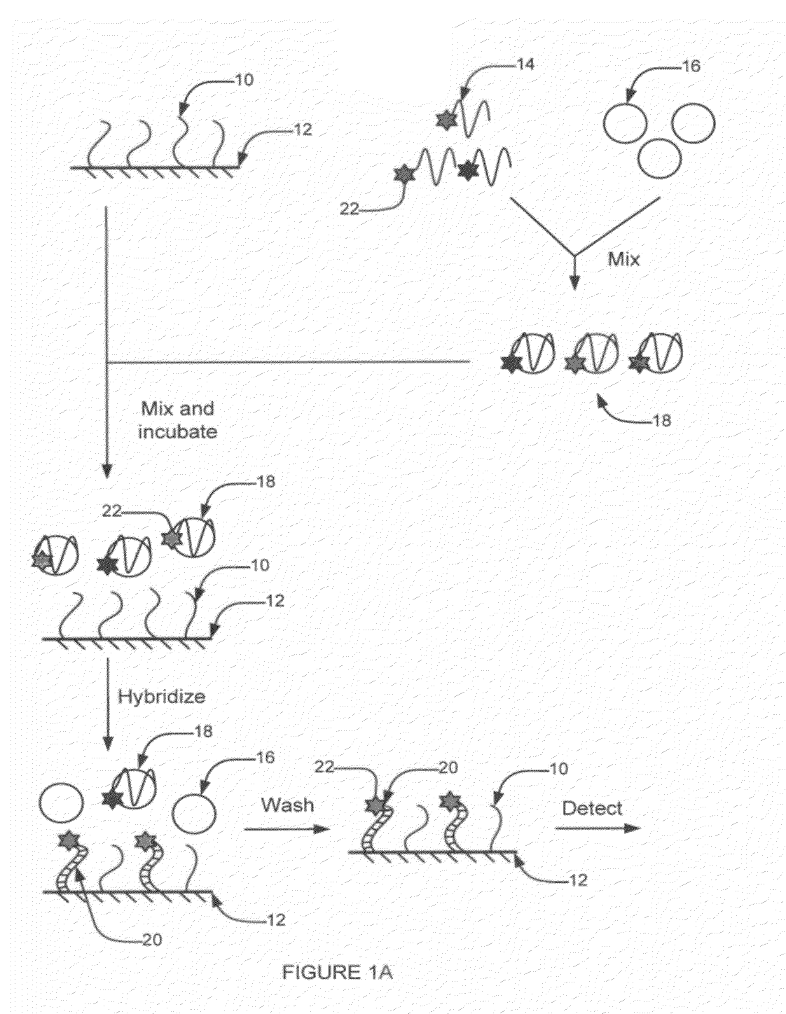

An example embodiment is schematically illustrated in FIG. 1. In a first step, probe nucleic acid molecules 10 are immobilized on a surface 12. In a second step, target nucleic acid molecules 14 are mixed with nanoparticles 16 to form complexes 18 comprising nanoparticles 16 non-covalently associated with target nucleic acid molecules 14. As described further below, nanoparticles 16 may comprise gold nanoparticles, for example. Nanoparticles 16 should be in suspended, non-aggregated form, or deaggregated under suitable conditions. In a third step, complexes 18 comprising nanoparticles 16 non-covalently associated with target nucleic acid molecules 14 are mixed with the probe nucleic acid molecules 10 immobilized on surface 12 and incubated for a period of time. As described further below, the incubation may be performed at ambient temperature or room temperature, for example. In a fourth step, a determination is made as to whether at least some of target nucleic acid molecules 14 have hybridized with probe nucleic acid molecules 10 to form a hybridization duplex 20 comprising a strand from target nucleic acid molecules 14 and a strand from probe nucleic acid molecules 10 and the level of hybridization. Each of these step are described in further detail below.

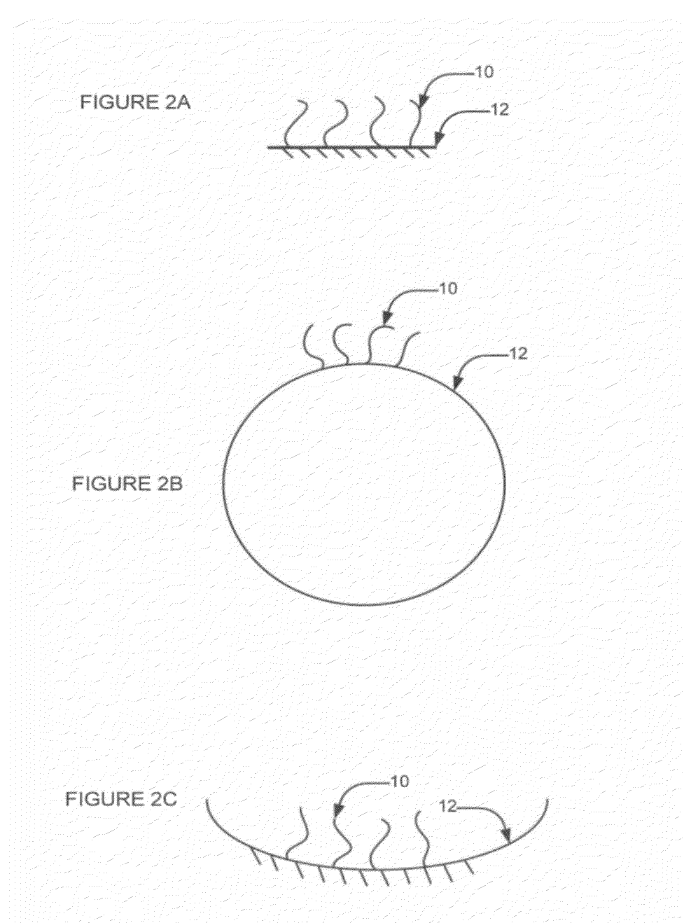

In the first step of FIG. 1, probe nucleic acid molecules 10 are immobilized on surface 12. Surface 12 may be a solid surface, or a semi-solid surface. For example, surface 12 may be a gel, polyacrylamide, agar, agarose, or gelatin. Surface 12 may be made of solid or curable materials, for example, glass, silicon, plastic, polymer, cellulose, etc. The solid surface may be, for example, a solid surface inside a test tube or a microfluidic channel, or on a glass slide, a test chip, a microarray chip, a microtiter plate, a nylon membrane, a film, or a head. Surface 12 may be substantially flat (see FIG. 2A), or curved, for example, the surface of a spherical bead (see FIG. 2B) or a well of a microtiter plate (see FIG. 2C). Surface 12 may also be coated or conjugated with one or more compounds, for example, surface 12 may be aldehyde-functionalized. If surface 12 is a solid surface on a bead, the bead may be magnetic or magnetically attractable.

There are a number of ways to immobilize or tether probe nucleic acid molecules 10 on surface 12. One approach is in situ synthesis, wherein probe nucleic acid molecules 10 are synthesized directly base by base on surface 12. Another approach is to spot or print probe nucleic acid molecules 10 on surface 12 using contact or non-contact printing methods. Other methods of immobilizing probe nucleic acid molecules 10 are known to persons skilled in the art. For example, immobilization can be achieved by chemical, mechanical, or biochemical methods such as covalent binding, adsorption, polymer encapsulation and so forth. As described further below, one example method of chemical immobilization is Schiff-base linkage formed between an aminated DNA or oligonucleotides probe and an aldehyde-functionalized glass surface.

Probe nucleic acid molecules 10 are typically single-stranded, or comprise at least a single-stranded region. In some embodiments, probe nucleic acid molecules 10 may also comprise a double-stranded region, or a triple-stranded region. Probe nucleic acid molecules 10 may be formed from oligonucleotides, DNA, RNA, or peptide nucleic acid (PNA). They may include both natural or artificial or synthetic nucleic acids. They may include genomic DNA or even a chromosome preparation (e.g., a chromosome preparation suitable for fluorescent in situ hybridization (FISH)). They may be synthesized or generated or amplified using standard procedures known to those skilled in the art or ordered from commercial vendors. Standard molecular biology methods for probe preparation can be found in Sambrook and Russel. Molecular Cloning: A Laboratory Manual, Cold Spring Harbor Laboratory Press, 2001, 3^{rd} edition, which is hereby incorporated by reference.

In some embodiments, probe nucleic acid molecules 10 may comprise DNA fragments having a length in the 1 Kb to 50 Kb range. In some embodiments, probe nucleic acid molecules 10 are between 10 and 1000 nucleotides in length. In some embodiments, probe nucleic acid molecules 10 are between 10 and 100 nucleotides in length. In some embodiments, probe nucleic acid molecules 10 are between 10 and 50 nucleotides in length. For oligonucleotides probes that are synthesized in situ, the maximum length is typically 80 to 100 nucleotides. For probes that are not synthesized in situ, they can be more than 1000 nucleotides in length.

In the second step of FIG. 1, a sample solution containing, complexes 18 comprising nanoparticles 16 non-covalently associated with target nucleic acid molecules 14 is provided. To prepare the sample solution, a first solution comprising target nucleic acid molecules 14 is mixed with a second solution comprising suspended non-aggregated nanoparticles 16. Target nucleic acid molecules 14 may be oligonucleotides, or DNA, or RNA, or PNA. Target nucleic acid molecules 14 may be single-stranded or double-stranded, or combinations thereof. As described in the background section, single-stranded nucleic acid molecules would bind non-covalently to nanoparticles, and double-stranded nucleic acid molecules would not bind to nanoparticles.

In some embodiments, a heat denaturing step is performed after target nucleic acid molecules 14 are mixed with nanoparticles 16. This is typically done if target nucleic acid molecules 14 are double-stranded, or comprise secondary structures (e.g., hairpins). For example, target nucleic acid molecules 14 can be denatured at an elevated temperature sufficient to separate them into single-stranded molecules or to remove the secondary structures so that target nucleic acid molecules 14 can non-covalently associate with or adsorb to the nanoparticles. For example, target nucleic acid molecules 14 may be denatured at a temperature between 85 to 100° C., for a period of time between 10 seconds to 5 minutes. For example, target nucleic acid molecules 14 may be denatured at 95° C. for 3 minutes. The incubation at the elevated temperature may promote the binding of single-stranded nucleic acid molecules 14 to nanoparticles 16. After formation of complex 18, the reaction solution may be snap chilled in an ice-water bath (e.g. at 4° C.) to prevent renaturation of single-stranded molecules or reformation of secondary structures.

The principle behind the non-covalent association between nanoparticles and single-stranded nucleic acid is briefly described in the background section above. Without wishing to be bound by any particular theory, it is likely that uncoiled single-stranded nucleic acid molecules hind or adsorb to nanoparticles through hydrophobic forces and/or van der Waals forces between the bases of the uncoiled single-stranded nucleic acid molecules and the nanoparticles. In contrast, double-stranded nucleic acid molecules do not bind to nanoparticles because the bases of the double-stranded nucleic acid molecules are not exposed and therefore not available for the aforementioned hydrophobic or van der Waals interactions. The non-covalent binding of single-stranded nucleic acid molecules to nanoparticles can be relatively tight and stable. However, this binding is reversible, and single-stranded nucleic acid molecules may dissociate from the nanoparticles under suitable conditions. As described further below, single-stranded nucleic acid molecules non-covalently bound to nanoparticles may dissociate from the nanoparticles and hybridize with complementary single-stranded nucleic acid molecules to form duplexes.

It should be emphasized that the binding between the nanoparticles and the single-stranded nucleic acid molecules in the present invention does not require covalent links between the nanoparticles and the single-stranded nucleic acid molecules. Therefore, it is not necessary for nanoparticles 16 to be covalently functionalized. Nor does it require target nucleic acid molecules 14 to be thiol-modified in order to be bound to nanoparticles 16.

Nanoparticles 16 may be sized between 1 and 100 nanometers. They may be spherical or rod-shaped or of other shapes. Nanoparticles 16 may be coated with negatively charged ions. As mentioned earlier, the negatively charged ions may help prevent aggregation of nanoparticles 16. Nanoparticles 16 may be formed of a metal, a semiconductor, or an uncharged substrate, such as glass, or combinations thereof. Nanoparticles 16 may be sized between 1 and 50 nm, or between 20 and 30 nm, or between 10 to 20 nm, or between 1 to 10 nm, or between 3.0 to 5.5 nm. Nanoparticles 16 may have a mean particle size of 5.0 nm. Nanoparticles may have a coefficient of variance of particle size that is less than 15% of the mean particle size.

Metal nanoparticles 16 may be formed of a conductive metal or metal alloy that allows a nanoparticle 16 to be capable of non-covalently associating with a single-stranded nucleic acid molecule. Prior to use in the present invention, it should be appreciated that the colloidal suspension should maintain the metal nanoparticles in a stable environment in which they are substantially free of aggregation. The metal nanoparticles should not significantly associate with double-stranded nucleic acid molecules. Example metal nanoparticles include, without limitation, gold nanoparticles, silver nanoparticles, platinum nanoparticles, mixed metal nanoparticles (e.g., gold shell surrounding a silver core), and combinations thereof. The metal nanoparticles may be magnetic or magnetically attractable, for example, formed of an inner core such as cobalt and an outer layer such as gold.

Preparation of colloidal metal nanoparticle suspensions can be carried out according to known procedures, e.g., Grabar et al. Anal. Chem. 67:735-743 (1995), which is hereby incorporated by reference in its entirety. Metal nanoparticles may be stabilized in the solution by negatively charged anions, such as citrate, acetate, carbonate, phosphate, oxalate, sulfate, or nitrate.

In some embodiments, nanoparticles 16 comprise gold nanoparticles. Preparation of Gold Nanoparticles can be Carried Out According to Known procedures, e.g., J. Turkevich, P. C. Stevenson, J. Hillier, Discuss. Faraday. Soc. 1951, 11, 55-75: J. Kimling, M. Maier, B. Okenve, V. Kotaidis, H. Ballot. A. Plech. J. Phys. Chem. B 2006, 110, 15700-15707; G. Frens, Colloid & Polymer Science, 1972, 250, 736-741; G. Frens, Nature (London). Phys. Sci. 1973, 241, 20-22; J. W. Slot and H. J. Geuze, Eur. J. Cell Biol. 38, 87 (1985); M. C. Daniels and D. Astruc. Chem. Rev. (Washington D.C.) 104, 293 (2004), each of which is hereby incorporated by reference in its entirety. Briefly, gold nanoparticles are typically produced in a liquid by reduction of chloroauric acid (HAuCl₄). After dissolving HAuCl₄, the solution is rapidly stirred while a reducing agent is added. This causes Au(III) ions to be reduced to neutral gold atoms. As more and more of these gold atoms form, the solution becomes supersaturated, and gold gradually starts to precipitate in the form of sub-nanometer particles. The rest of the gold atoms that form stick to the existing particles, and, if the solution is stirred vigorously enough, the particles will be fairly uniform in size. The anions in gold nanoparticle preparation also prevent the gold nanoparticles from aggregating. These anions may include citrate, acetate, carbonate, phosphate, oxalate, sulfate, or nitrate.

Alternatively, nanoparticles 16 can be purchased from commercial sources. For example, gold nanoparticles can be purchased from Sigma Life Sciences. The gold nanoparticles from Sigma Life Sciences may be in aqueous solution and may have a particle size in the range of 3.0 to 5.5 nm and a mean particle size of 5.0 nm.

Nanoparticles 16 may also be formed of an uncharged substrate (e.g. glass). The substrate may be charged using anions or polyanions. The anions or polyanions can be coupled to the substrate (e.g., glass) using standard glass binding chemistry. Example anions include, without limitation, citrate, acetate, carbonate, dihydrogen phosphate, oxalate, sulfate, and nitrate. Example polyanions include, without limitation, poly(2-acrylamido-2-methyl-1-propanesulfonic acid), poly(acrylic acid), poly(anetholesulfonic acid), poly(anilinesulfonic acid), poly(sodium 4-styrenesulfonate), poly(4-styrenesulfonic acid), and poly(vinylsulfonic acid). Other anions and polyanions may also be employed.

Although some of the examples in the present disclosure describe experiments performed using gold nanoparticles, it will be appreciated by those skilled in the art that other nanoparticles having similar properties may also be used. For example, silver nanoparticles have been shown to behave like gold nanoparticles in terms of non-covalent binding with single-stranded nucleic acid molecules: Chen et al., Analyst, (2010), 135, 1066-1069, which is hereby incorporated by reference.

Target nucleic acid molecules 14 may be oligonucleotides, deoxyribonucleic acid (DNA), ribonucleic acid (RNA) or an artificial or synthetic nucleic acid. They may be synthesized or generated or amplified using standard procedures known to those skilled in the art or ordered from commercial vendors. Target nucleic acid molecules 14 may be isolated directly from samples (e.g. cells, tissues, cell extracts, tissue culture media, bodily fluids, environmental samples, other biological samples etc.), or they may first be amplified by polymerase chain reaction (PCR) or reverse-transcription PCR(RT-PCR). Target nucleic acid molecules 14 may comprise synthetic, natural, or structurally Modified nucleoside bases. Target nucleic acid molecule 14 can also be from any source organism (e.g., human or another animal, virus, bacteria, insect, plant, etc.).

Target nucleic acid molecules 14 may be of any length that is suitable for non-covalent association with nanoparticles. In some embodiments, target nucleic acid molecules 14 are between 10 to 1000 nucleotides or base-pairs in length. In some embodiments, target nucleic acid molecules 14 may exceed 1000 nucleotides or base-pair in length. Target nucleic acid molecules 14 may comprise single-stranded molecules, or double stranded molecules, or combinations thereof. In some embodiments, target nucleic acid molecules 14 may comprise a single-stranded region and a double-stranded region. Target nucleic acid molecules 14 may be purified or isolated molecules, or may be present in a solution or sample that comprises other molecules or contaminants. Target nucleic acid molecules 14 may comprise nucleic acid molecules having different sequences (e.g., a mixture of genomic DNA molecules, a mixture of different PCR products, a mixture of cDNA molecules, or a mixture comprising two related DNA sequences differing by a single base-pair). Target nucleic acid molecules 14 may be from a single sample source or may be from two or more sample sources (e.g., pooled cDNA molecules from two types of cells, one being stem cell, the other being differentiated cell, or genomic DNA from two human individuals).

Target nucleic acid molecules 14 can either be unlabeled or they can be conjugated or otherwise coupled to a detectable label 22. FIG. 1A shows a method that is the same as the method illustrated in FIG. 1 except that target nucleic acid molecules 14 are labeled with detectable label 22. Suitable detectable labels 22 include, without limitation, fluorescent labels, redox (electrochemical) labels, and radioactive labels.

Coupling of a fluorescent label to nucleic acid molecules can be achieved using known nucleic acid-binding chemistry or by physical means, such as through ionic, covalent or other forces well known in the art (see, e.g., Dattagupta et al., Analytical Biochemistry 177:85-89 (1989): Saiki et al. Proc. Natl. Acad. Sci. USA 86:6230-6234 (1989); Gravitt et al. J. Clin. Micro. 36:3020-3027 (1998), each of which is hereby incorporated by reference in its entirety). Either a terminal base or another base near the terminal base can be bound to the fluorescent label. For example, a terminal nucleotide base of the target nucleic acid molecules can be modified to contain a reactive group, such as (without limitation) carboxyl, amino, hydroxyl, thiol, or the like.

The fluorescent label can be any fluorophore that can be conjugated to a nucleic acid and preferably has a photoluminescent property that can be detected and easily identified with appropriate detection equipment. Exemplary fluorescent labels include, without limitation, fluorescent dyes, semiconductor quantum dots, lanthanide atom-containing complexes, and fluorescent proteins. Example fluorescent dyes include, without limitation. Calcein. FITC, Alexa™, Rhodamine 110, 5-FAM, Oregon Green™ 500, Oregon Green™ 488, RiboGreen™, Rhodamine Green™, Rhodamine 123, Magnesium Green™, Calcium Green™, Cy3™, Alexa™ 546, TRITC, Magnesium Orange™, Phycoerythrin R&B, Rhodamine Phalloidin, Calcium Orange™, Pyronin Y, Rhodamine B, TAMRA, Rhodamine Red™, ROX, Nile Red, YO-PRO™-3. R-phycocyanin, C-Phycocyanin, Cy5™, Thiadicarbocyanine, and Cy5.5™. Other dyes now known or hereafter developed may similarly be used.

The molar ratio of single-stranded target nucleic acid molecules 14 to nanoparticles 16 can be varied within relatively wide limits. In some embodiments, the molar ratio of single-stranded target nucleic acid molecules 14 to nanoparticles 16 may be in the range of 4 to 1. For example, the molar ratio may be 4:1, 3:1, 2:1, or 1:1. As described further below, a lower molar ratio may result in a higher degree of discrimination between perfect match (PM) and single-base-pair mismatch (MM) sequences in a hybridization reaction.

In some embodiments, complexes 18 comprising nanoparticles 16 non-covalently associated with single-stranded target nucleic acid molecules 14 are used immediately after formation for hybridization with probe nucleic acid molecules 10 immobilized on surface 12, as described later. In some embodiments, complexes 18 may be stored at a low temperature (e.g., 4° C.) for future use. For example, complexes 18 comprising gold nanoparticles 16 non-covalently associated with single-stranded target nucleic acid molecules 14 remain stable at 4° C. for at least 48 hours. In some embodiments, sample solutions containing complexes 18 may be diluted to a desired concentration before hybridization.

In the third step of FIG. 1, the sample solution containing complexes 18 comprising nanoparticles 16 non-covalently associated with single-stranded target nucleic acid molecules 14 are mixed with probe nucleic acid molecules 10 immobilized on surface 12 and incubated for a period of time. Because the non-covalent binding between a nanoparticle and a single-stranded nucleic acid molecule is relatively stable, a single-stranded target nucleic acid molecule would typically only dissociate from the nanoparticle if the target nucleic acid molecule has a sequence that is complementary to the sequence of a probe nucleic acid molecule and the probe nucleic acid molecule is available to hybridize with the target nucleic acid molecule. If the target nucleic acid molecule has a sequence that is perfectly complementary (i.e., a perfect match) to the sequence of the immobilized probe nucleic acid molecule, the target nucleic acid molecule would tend to dissociate from the nanoparticles and hybridize with the immobilized probe nucleic acid molecule. If the target nucleic acid molecule has a sequence that is non-complementary and unrelated to the sequence of the immobilized probe nucleic acid molecule, the target nucleic acid molecules would tend to remain associated with the nanoparticles and not hybridize with the immobilized probe nucleic acid molecule. If the target nucleic acid molecule has a sequence that is related to the sequence of the immobilized probe nucleic acid molecule but has one or a few base-pair mismatches to the sequence of the immobilized probe nucleic acid molecule, the target nucleic acid molecules would not tend to dissociate from the nanoparticles and hybridize with the immobilized probe nucleic acid molecule.

In other words, nanoparticles 16 serve as a carrier or competitor in the hybridization process, and probe nucleic acid molecules 10 and nanoparticles 16 compete for binding with target nucleic acid molecules 14. Since binding of either probe nucleic acid molecules 10 or nanoparticles 16 with target nucleic acid molecules 14 are reversible processes, the amount of target nucleic acid molecules 14 that would dissociate from nanoparticles 16 and hybridize with probed nucleic acid molecules 10 would largely depend on the thermodynamic stability of hybridization duplexes 20. Typically, hybridization duplexes composed of two perfectly matched strands are thermodynamically more stable (i.e., having lower Gibbs free energy) than hybridization duplexes composed of two mismatched strands. The nanoparticles therefore could be used to discriminate between nucleic acid molecules having a sequence complementary to the sequence of probe nucleic acid molecules and nucleic acid molecules having a sequence not complementary to the sequence of the probe nucleic acid molecules.

In some embodiments, the hybridization step is performed at elevated temperatures (e.g., above ambient temperature or room temperature). For, example, the hybridization step may be performed at a temperature between 30 to 70° C., and in an incubation oven. However, this is not necessary. In some embodiments, the hybridization step is performed at ambient temperature or room temperature without the use of any specialized equipment for temperature control. For example, the hybridization step may be performed at a temperature between 20 to 30° C. for example, between 22 and 26° C. or between 22 and 24° C., or between 24 and 26° C.

As described further later, the method provides a high degree of discrimination between nucleic acid molecules having a sequence complementary (i.e., perfect match) to the sequence of probe nucleic acid molecules and nucleic acid molecules having a sequence that has a single-base-pair mismatch to the sequence of the probe nucleic acid molecules without the use of elevated temperatures or a heating equipment.

Buffer conditions for hybridization are well known to those skilled in the art and can be varied within relatively wide limits. The term hybridization stringency refers to the degree to which hybridization conditions disfavor the formation of hybrids containing mismatched nucleotides, thereby promoting the formation of perfectly matched hybrids; with higher stringency correlated with a lower tolerance for mismatched hybrids. Factors that affect the stringency of hybridization include, but are not limited to, pH, ionic strength, concentration of organic solvents such as formamide and dimethylsulfoxide, and concentration of sodium dodecyl sulfate (SDS). As is well known to those of skilled in the art, hybridization stringency is increased by higher temperatures, lower ionic strengths, and lower solvent concentrations. See, for example, M. A. Innis et al. (eds.) PCR Protocols, Academic Press, San Diego, 1990: B. D. flames et al. (eds.) Nucleic Acid Hybridisation: A Practical Approach, IRL Press, Oxford, 1985, which are hereby incorporated by reference in their entirety.

in some embodiments, the hybridization buffer comprises 1.0× saline-sodium citrate (SSC) and 0.2% sodium dodecyl sulfate (SDS). It will be appreciated by those skilled in the art that buffers comprising very high salt (e.g. Na⁺) concentrations should typically be avoided. For example, buffers comprising Na⁺ concentrations greater than 0.3 M should typically be avoided. This is to prevent the premature aggregation of the nanoparticles, although nanoparticle aggregation is typically inhibited by the non-covalently associated single-stranded nucleic acid molecules. In other words, single-stranded nucleic acid molecules stabilize the nanoparticles in the suspended, non-aggregated form. It will be appreciated by those skilled in the art that many other hybridization buffers can also be used.

After incubation, surface 12 with immobilized probe nucleic acid molecules 10 and hybridization duplexes 20, if any, is washed with a wash solution. The wash buffer may be the same or different from the hybridization buffer. The wash step washes away target nucleic acid molecules 14 that remain associated with nanoparticles 16 as well as nanoparticles 16. Target nucleic acid molecules 14 that have hybridized with probe nucleic acid molecules 10 as hybridization duplexes 20 would remain on surface 12.

In the fourth step of FIG. 1, a determination is made as to whether at least some of target nucleic acid molecules 14 have hybridized with probe nucleic acid molecules 12 to form hybridization duplexes 20 and the level of hybridization. In some embodiments, surface 12 is dried before a detection method is applied. In some embodiments, a detection method may be applied without drying surface 12.

This determination or detection may be qualitative or quantitative. A large number of methods are available to detect or quantify hybridization duplexes 20 on surface 12. For example, if target nucleic acid molecules 14 were fluorescently labeled prior to mixing and incubation (FIG. 1A), surface 12 can be scanned for fluorescence emissions. For example, a confocal laser fluorescent scanner may be used. If both the target and the probe are fluorescently labeled, a detection method called fluorescence resonance energy transfer (FRET) may be used. If the probe is a chromosome preparation, detection methods suitable for fluorescent in situ hybridization (FISH) may be used. Alternatively, if the target nucleic acid molecules comprise redox labels, or radioactive labels, other methods may be used to detect the level of hybridization. These methods are well known to those skilled in the art.

### Distinguishing Between Two Different Nucleic Acid Molecules

The above described nanoparticle assisted hybridization method can be used to distinguish between two different nucleic acid molecules. In some embodiments, the method can be used to distinguish between two related nucleic acid sequences. In some embodiments, the method can be used to distinguish between two related nucleic acid sequences that differ by two or more nucleotides. In some embodiments, the method can be used to distinguish between two related nucleic acid sequences that differ by a single nucleotide. It is often advantageous to distinguish between two or more nucleic acid sequences which are related but which differ by a single nucleotide. For example, single nucleotide polymorphisms (SNPs) in human and other mammalian genomes are characterized by a sequence difference of a single nucleotide. Also, many mutations of clinical significance differ by only a single nucleotide.

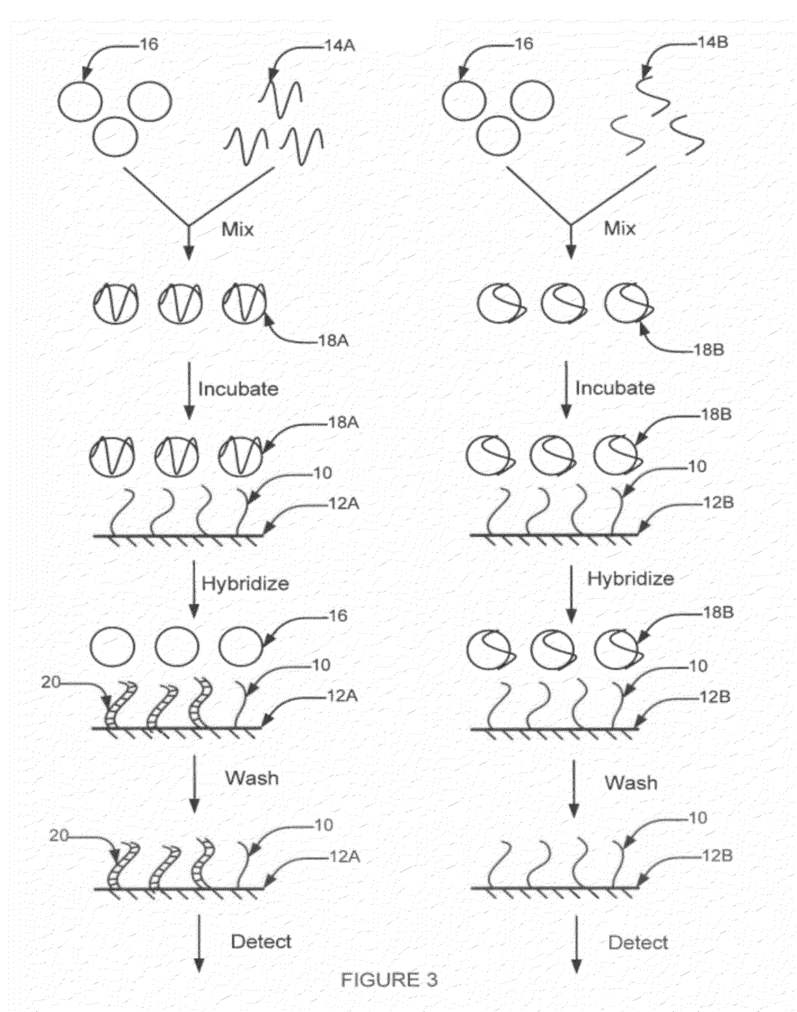

One example embodiment is described here, which is a method for distinguishing between two different nucleic acid samples. As illustrated in FIG. 3, the method involves performing two nanoparticle-assisted hybridization assays in parallel with two different nucleic acid samples 14A, 14B. Since the method is based on the already described nanoparticle-assisted hybridization method, only key steps are described below and the reader can refer to the previous section for further details.

First, probe nucleic acid molecules 10 are provided and immobilized on a first surface 12A and a second surface 12B. Typically, an equal amount of the probe nucleic acid molecules 10 are immobilized on first surface 12A and second surface 12B.

Second, first and second nucleic acid samples 14A and 14B are mixed separately with nanoparticles 16 under identical or comparable conditions to form nucleic acid-nanoparticle complexes 18A and 18B. As described earlier, only single-stranded nucleic acid molecules bind or adsorb to nanoparticles 16. Therefore, if the first and second nucleic acid samples 14A and 14B are double-stranded, they need to be heat denatured into single-stranded molecules. These two parallel reactions should be performed under identical or comparable conditions. In some embodiments, the amounts and concentrations of the first and second nucleic acid samples 14A and 14B are comparable. In some embodiments, one of the nucleic acid samples 14A and 14B may be a positive control, i.e., having a sequence that is known to be complementary to the sequence of the probe nucleic acid molecules 10. In some embodiments, one of the nucleic acid samples 14A and 14B may be a negative control, i.e., having a sequence that is known to be not complementary or unrelated to the sequence of the probe nucleic acid molecules 10.

Third, the two complexes 18A and 18B from the previous step are separately mixed and incubated with the immobilized nucleic acid probe 10 in a suitable hybridization buffer. If one of the nucleic acid samples 14A and 14B has a sequence that is complementary to the sequence of the probe nucleic acid molecules 10, it would dissociate from nanoparticles 16 and hybridize with probe nucleic acid molecules 10. As mentioned before, this step may be performed at room temperature (e.g., 22-26° C.), because of the higher level of discrimination provided by the nanoparticles against hybridization between mismatched nucleic acid molecules. After the incubation is completed, surfaces 12A, 12B with the immobilized probe nucleic acid molecules 10 and hybridization duplexes 20, if any, are washed with a wash buffer. These two parallel reactions should be performed under identical or comparable conditions.

Fourth, determinations are made as to how much of the first nucleic acid sample 14A and how much of the second nucleic acid 14B sample is hybridized with probe 10. The results can be used to infer if one of the nucleic acid samples 14A, 14B is complementary or non-complementary to probe 10.

In other words, two parallel and separate nanoparticle-assisted hybridization reactions are carried out, one using a first nucleic acid sample 14A and a probe 10, the other using a second nucleic acid sample 14B and the same probe 10, under identical or comparable conditions.

The inventors have determined, as described further later, that use of nanoparticles increases the discrimination ratio between a perfectly matched (PM) sequence and a single base-pair mismatch (MM) sequence. In the present disclosure, the term “discrimination ratio” refers to the ratio between the degree of hybridization of the PM sequence with the probe and the degree of hybridization of the MM sequence with the probe. When both the PM and MM sequences are fluorescently labeled, the discrimination ratio may be calculated based on the ratio between the measured fluorescence for the PM sequence and the measured fluorescence for the MM sequence upon hybridization with the probe.

Because use of nanoparticles increases the discrimination ratio, it is possible to perform the incubation step at a temperature that is lower than conventional hybridization or DNA microarray protocols and achieve the same degree of discrimination. In some embodiments, the hybridization step is performed at ambient temperature or room temperature without the use of any specialized equipment for temperature control. For example, the hybridization step may be performed at a temperature between 20 to 30 degrees Celsius, for example, between 22 and 26 degrees Celsius, or between 22 and 24 degrees Celsius, or between 24 and 26 degrees Celsius. Under these ambient or room temperature incubation conditions, the discrimination ratio may be greater than 5:1, for example, greater than 10:1, or 15:1, or 20:1, or 25:1.

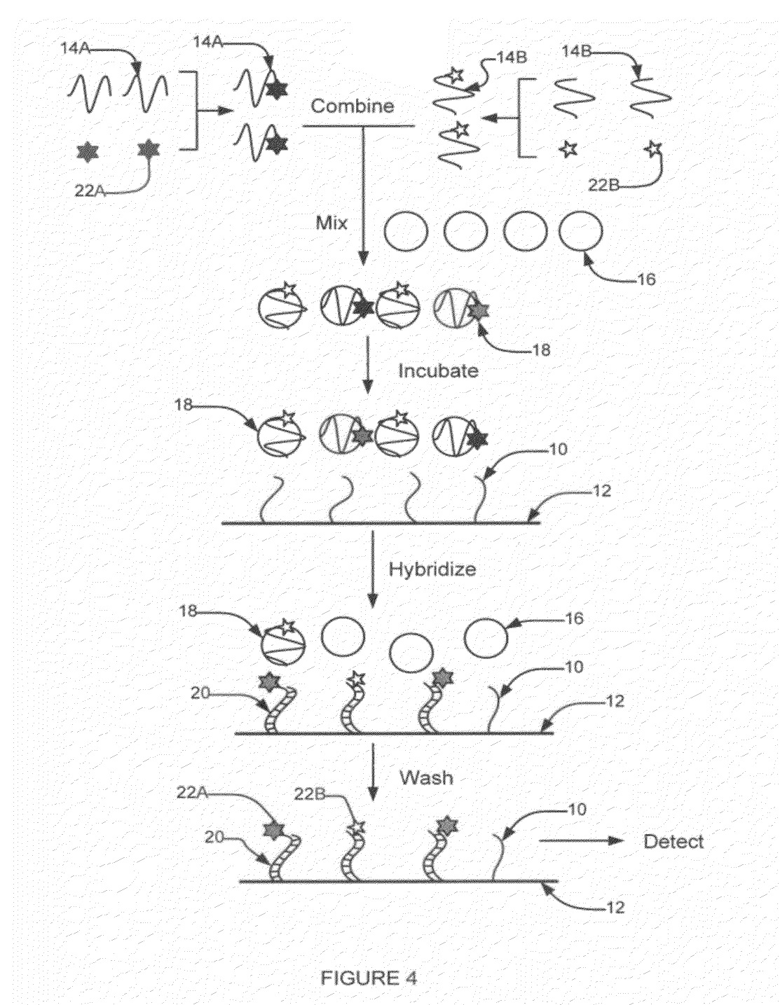

Another example embodiment is described below, which is also a method for distinguishing two different nucleic acid samples and is also a variation of the earlier described nanoparticles-assisted hybridization method. As illustrated in FIG. 4, the method involves incubating two different nucleic acid samples in a single hybridization solution.

First, probe nucleic acid molecules 10 are provided and are immobilized on a surface 12 as described above.

Second, the first and second nucleic samples 14A, 14B are separately labeled with two respective detectable labels 22A, 22B (e.g., two different fluorescent labels). For example, first nucleic acid sample 14A may be labeled with Cy3 and second nucleic acid sample may be labeled with Cy5. Then the first and second nucleic samples 14A, 14B are mixed in a single sample solution. The amounts and concentrations of the first and second nucleic acid samples 14A, 14B should be comparable. In some embodiments, there is an equal molar concentration of the first and second nucleic acid samples 14A, 14B in the sample solution. In some embodiments, one of the nucleic acid samples 14A and 14B may be a positive control, i.e., having a sequence that is known to be complementary to the sequence of the probe nucleic acid molecules 10. In some embodiments, one of the nucleic acid samples 14A and 14B may be a negative control, i.e., having a sequence that is known to be not complementary or unrelated to the sequence of the probe nucleic acid molecules 10.

The sample solution is then incubated with the immobilized probe nucleic acid molecules 10 in a single reaction. As mentioned before, the incubation can be performed at room temperature.

After incubation and washing, a determination is made as to how much of the first sample nucleic acid 14A and how much of the second sample nucleic acid 14B is hybridized with the probe 10. For example, this can be determined by measuring the intensity of the two fluorescent labels. The results can be used to infer if one of the nucleic acid samples 14A, 14B is complementary or non-complementary to probe 10.

When two nucleic acid samples 14A, 14B are mixed in a single solution and each is conjugated to a different fluorescent label 22A, 22B, it is preferable that the fluorescent labels 22A, 22B can be distinguished from one another using appropriate detection equipment. That is, the fluorescent emissions of one fluorescent label should not overlap or interfere with the fluorescent emissions of another fluorescent label. For example, one fluorescent label may be Cy3 and the other fluorescent label may by Cy5. Other example pairs of fluorescent labels that are suitable for this purpose are well-known to those skilled in the art.

Use of Nanoparticle-Assisted Hybridization with Microarrays

The nanoparticle-assisted hybridization method can also be applied to microarray technology. A DNA microarray is a multiplex technology commonly used in molecular biology. It consists of an arrayed series of tens, hundreds, thousands, or even tens of thousands of microscopic spots of picomoles (10⁻² moles) of oligonucleotides or DNA probes, each having a specific nucleotide sequence. These can be a short section of a gene or other DNA element that are used to hybridize a sample (e.g. cDNA or genomic DNA or RNA) under high-stringency conditions. Probe-target hybridization is usually detected and quantified by detection of fluorophore-, silver-, or chemiluminescence-labeled targets to determine relative abundance of nucleic acid sequences in the sample. Since an array can contain tens, hundreds, thousands, or even tens of thousands of probes, a microarray experiment can accomplish many genetic tests in parallel. Therefore arrays have dramatically accelerated many types of investigation.

In standard microarrays, the probes are typically attached via surface engineering to a solid surface by a covalent bond to a chemical matrix (via epoxy-silane, amino-silane, lysine, polyacrylamide or others). The solid surface can be glass or a silicon chip, for example, in microarray chips produced by Affymetrix. Other microarray platforms, such as Illumina, may use microscopic beads, instead of the large solid support.

DNA microarrays can be used to measure changes in gene expression levels, to detect single nucleotide polymorphisms (SNPs), or to genotype or sequence mutant genomes. Procedures for using DNA microarrays are well-known to those skilled in the art: e.g., David Bowtell and Joseph Sambrook, DNA Microarrays: A Molecular Cloning Manual, Cold Spring Harbor Laboratory Press; 1st edition (2002), which is hereby incorporated by reference.

An example embodiment according to the present invention is described below. First, a surface 12 is provided. Surface 12 may be any surface suitable for microarrays. Surface 12 may comprise a glass slide, a glass chip, a compact disc, a plate, a membrane, a film, or beads, etc.

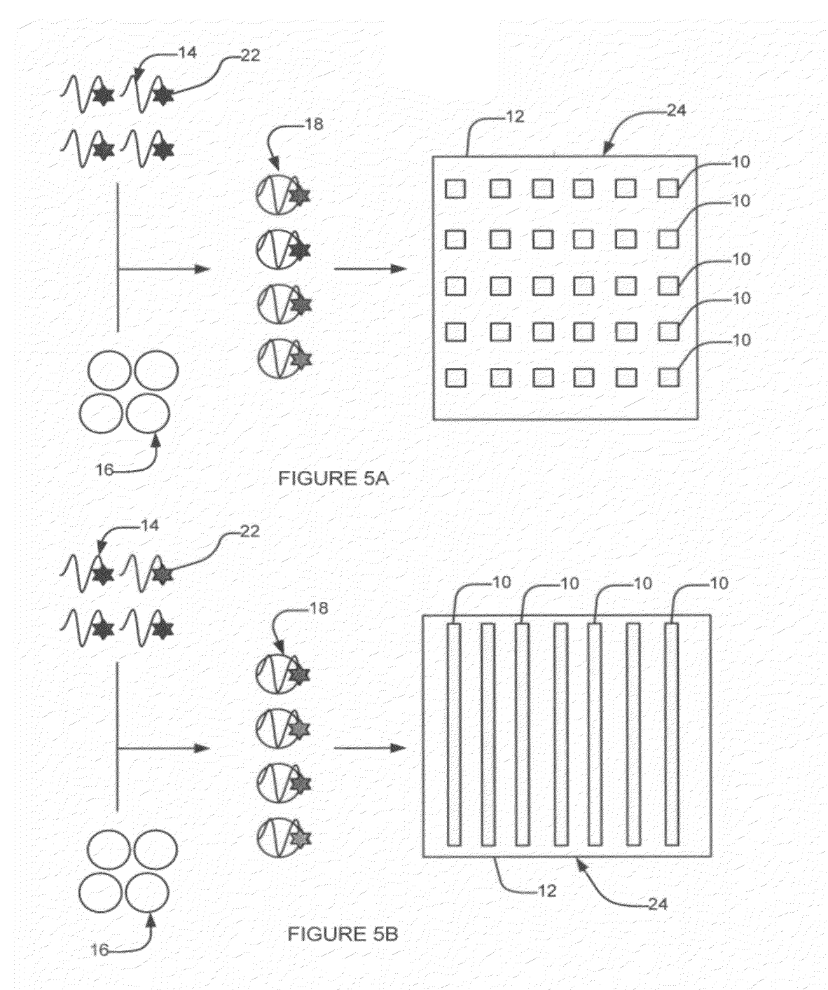

Second, a plurality of nucleic acid probes 10 is immobilized on surface 12 at spaced-apart positions. These positions may be discrete spots or lines or some other patterns. FIG. 5A shows an example microarray 24 having a dot array pattern. FIG. 5B shows an example microarray 24 having a line pattern. As mentioned before, there are a number of ways to immobilize or tether probe nucleic acid molecules 10 on surface 12. One approach is in situ synthesis, wherein the probe nucleic acid molecules 10 are synthesized directly base by base on surface 12. Another approach is to spot or print the probe nucleic acid molecules 10 on surface 12 using contact or non-contact printing methods. Another approach is chemical immobilization which is based on Schiff-base linkage formed between aminated probe and aldehyde-functionalized surface. This approach is described in detail later.

Third, a sample solution is prepared and incubated with the probes. The sample solution contains complexes 18 comprising sample nucleic acid molecules 14 non-covalently associated with nanoparticles 16. Sample nucleic acid molecules 14 may be obtained from a single sample source, or from two different sample sources (for example, a healthy cell and a diseased cell). If the sample nucleic acid molecules 14 are obtained from two different sources, they may be labeled with two different detectable labels 22 (e.g., two different fluorophores) and then mixed together. Sample nucleic acid molecules 14 are then mixed with nanoparticles 16 to form a sample solution. Sample nucleic acid molecules 14 may be heat denatured to separate into single-stranded molecules before non-covalently binding or adsorbing to nanoparticles 16.

Fourth, the sample solution is incubated with microarray 24. Because nanoparticles 16 can increase the discrimination ratio between perfectly matched and mismatched sequences, the requirement for thermal stringency is reduced or eliminated. In some embodiments, the hybridization step is performed at ambient temperature or room temperature. For example, the hybridization step may be performed at a temperature between 20 to 30 degrees Celsius, for example, between 22 and 26 degrees Celsius, or between 22 and 24 degrees Celsius, or between 24 and 26 degrees Celsius. Advantageously, the use of an incubation oven that provides elevated temperatures is not required. After incubation is completed, microarray 24 is washed to remove the nanoparticles 16 and un-hybridized nucleic acid molecules 14.

Fifth, after incubation and washing, a determination is made as to the level of hybridization with each of the probes 10. Suitable microarray scanners can be used and computer software can be used to analyze the microarray data. For example, the microarray data can be analyzed to infer gene expression levels, or the presence or absence of specific DNA sequences (e.g. SNP) in a sample.

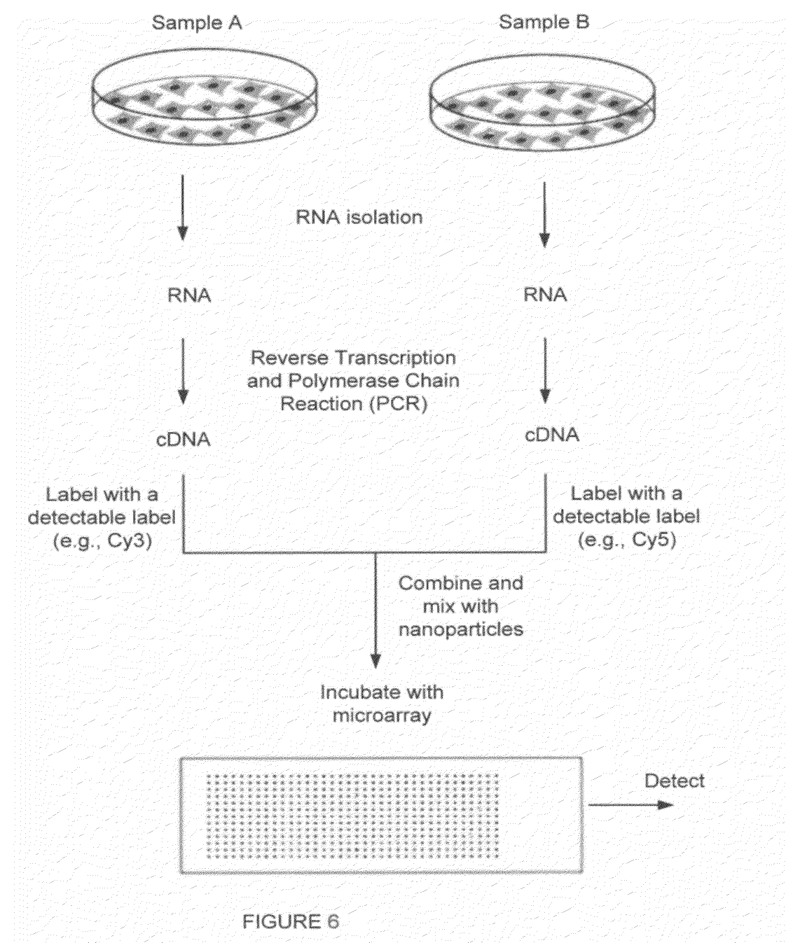

FIG. 6 illustrates an example embodiment, where the sample nucleic acid molecules are obtained from two separate sources. In FIG. 6, Sample A may be cancer cells, and Sample B may be normal cells. RNA is isolated from Sample A and Sample B and reverse transcription is performed to generate cDNA. The cDNA from Sample A is labeled with a first detectable label (e.g., Cy3), and the cDNA from Sample B is labeled with a second detectable label (e.g. Cy5). The two cDNA samples are then combined and mixed with nanoparticles, heat denatured, and then incubated with a microarray. As will be apparent to those skilled in the art, other forms of DNA or RNA or oligonucleotides samples may be used. For example, the sample DNA may comprise genomic DNA, mitochondrial DNA or chloroplast DNA.

One of the advantages of the present invention in microarray analysis is that the use of nanoparticles non-covalently associated with sample nucleic acid molecules could obviate the need to maintain high temperatures throughout the hybridization process, resulting in reduced cost and analysis time. In addition, the nanoparticle-assisted approach may normalize the reaction conditions across different probe/target pairs, allowing for high-throughput analysis of multiple DNA samples simultaneously. In contrast, in conventional microarray analysis, different probe/target pairs may require different optimal elevated hybridization temperature and a single optimal hybridization is not available across different probe/target pairs.

Use of Nanoparticle-Assisted Hybridization with MMA

One aspect of the invention provides nanoparticle-assisted hybridization methods in association with a microfluidic microarray assembly (MMA) or a microchannel plate assembly. MMA and microchannel plate assemblies are described in WO 2006/060922 and L. Wang and P. C. H. Li, J. Agric. Food. Chem. 55, 10509 (2007), which are hereby incorporated by reference in their entirety. It should be noted that both MMA and microchannel plate assembly can be considered to be a subset of microarrays, and that microchannel plate assembly can be considered to be a subset of MMA.

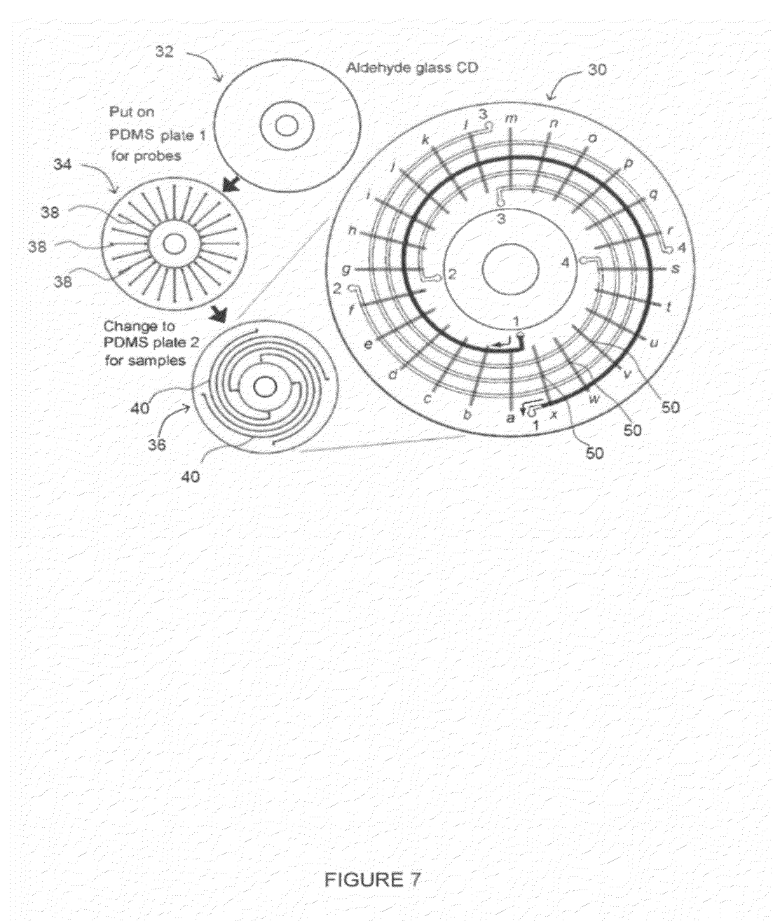

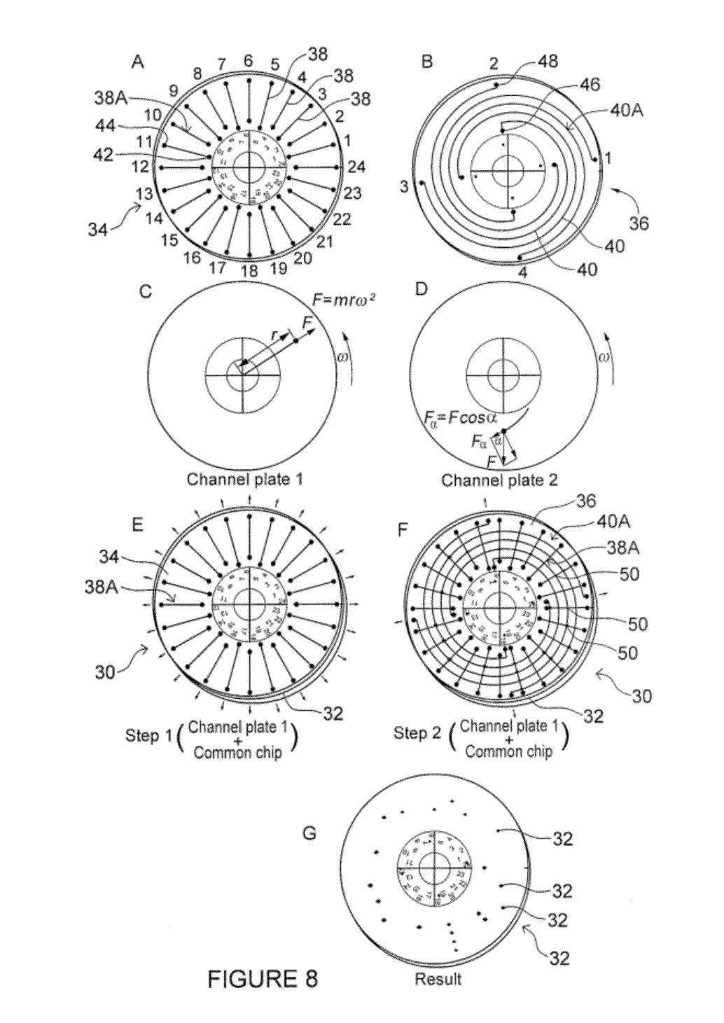

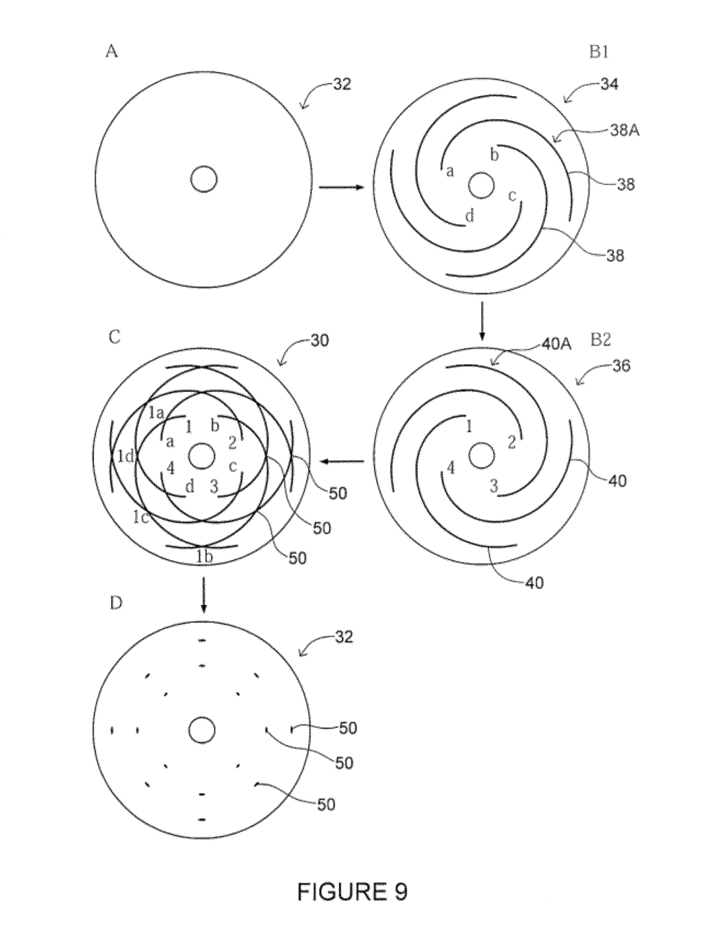

The general concept of an embodiment of MMA is shown in FIGS. 7 and 8. In this embodiment a microfluidic microarray assembly (MMA) 30 is illustrated which is produced by the combination of a test chip (“common chip”) 32 and a first channel plate 34 and/or a second channel plate 36. As described in detail below, channel plates 34, 36 may be each separately connected to test chip 32 in consecutive order, to deliver reagents to test chip 32 (such as probes or test samples) in predetermined patterns defined by microfluidic channel patterns. That is, in one example, first channel plate 34 is first sealingly connected to test chip 32 to deliver a plurality of probes thereto. First channel plate 34 is then removed from test chip 32 and second channel plate 36 is sealingly connected to test chip 32 to deliver a plurality of samples thereto. MMA thus enables the efficient formation of high density multi-probe, multi-sample microarrays by employing microfluidics.

As shown best in FIGS. 7 and 8, first channel plate 34 has a plurality of first microfluidic channels (or “microchannels”) 38 arranged in a first predetermined reagent pattern 38A, such as a radial pattern comprising a plurality of linear, radially extending segments. In the example of FIGS. 7 and 8, first channel plate 34 has 24 separate radially extending microfluidic channels 38. Similarly, second channel plate 36 has a plurality of second microfluidic channels 40 arranged in a second predetermined reagent pattern 40A, such as a spiral pattern. In the example of FIGS. 7 and 8, second channel plate 36 shown in FIGS. 7 and 8 has 4 separate spiral microfluidic channels 40.

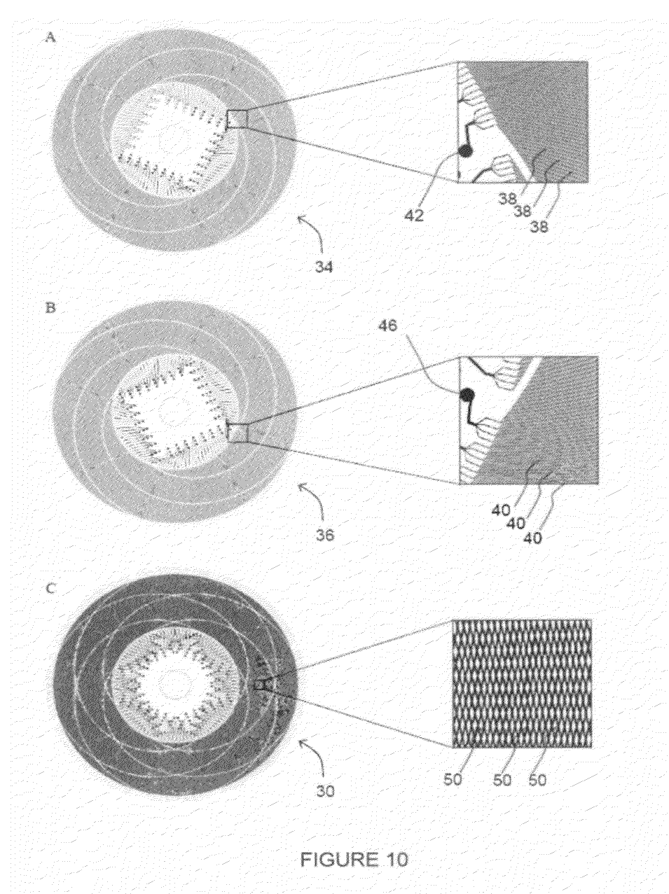

Reservoirs are located at each end of microfluidic channels 38, 40 in fluid communication therewith. More particularly, each first microfluidic channel 38 has an inlet reservoir 42 at one end thereof and an outlet reservoir 44 at the other end thereof and each second microfluidic channel 40 has an inlet reservoir 46 at one end thereof and an outlet reservoir 48 at the other end thereof (FIGS. 8A and 8B). In the case of high density microarrays, the inlet and/or outlet reservoirs may be staggered in rows to lit within the available space on MMA 30 (FIG. 10).

First and second predetermined reagent patterns 38A, 40A, and hence the geometric configurations of first and second microfluidic channels 38, 40, preferably differ. For example, first predetermined reagent pattern 38A may be a radial pattern and second predetermined reagent pattern 40A may be a spiral pattern, or vice versa. This results in an intersecting pattern of reagent deposition on test chip 32 when each of the channel plates 34, 36 is consecutively sealed to test chip 32 and reagents are flowed through microfluidic channels 38, 40.

For example, in FIGS. 7 and 8E, when first channel plate 34 is sealed with test chip 32, one or more first reagents can be loaded into inlet reservoirs 42 and flowed through first microfluidic channels 38 to outlet reservoirs 44. This results in the distribution of the first reagent in a radial pattern 38A on test chip 32. As described below, the first reagent is then immobilized on test chip 32 and first channel plate 34 is removed. Second channel plate 36 is then sealed to test chip 32 (FIGS. 7 and 8F). One or more second reagents are loaded into inlet reservoirs 46 and flowed through second microfluidic channels 40 to outlet reservoirs 48. This results in the distribution of the second reagent in a spiral pattern 40A on test chip 32. The intersection points between first and second predetermined patterns 38A, 40A (in this case the radial pattern and the spiral pattern) defines a plurality of microarray test positions 50 on test chip 32. If the first reagent reacts with the second reagent at select test positions 52, a positive test result is obtained (FIG. 8G). For example, as discussed further below, a positive test result could indicate reaction (e.g. hybridization) between the first reagent and the second reagent, formation of a reaction product, modification of a biochemical or cellular parameter or the like.

The number of microarray test positions 50 which are created from the intersection points of first and second predetermined reagent patterns 38A, 40A on test chip 32 depends upon the number and configuration of microfluidic channels 38, 40 on first and second channel plates 34, 36, respectively. For example, in this embodiment, each line of the first reagent pattern produced by first microfluidic channels 38 intersects only once with each line of the second reagent pattern produced by second microfluidic channels 40. Thus, if first channel plate 34 has x microfluidic channels 38 and second channel plate 36 has y microfluidic channels 40, the resulting microarray has x*y number of intersection points or test positions 50. In FIGS. 7 and 8, first channel plate 34 has x=24 radial microfluidic channels 38 and second channel plate 36 has y=4 spiral microfluidic channels 40. The resulting microarray has 24*4=96 test positions 50 on test chip 32. Preferably, there is only one intersection point between each line of the first reagent pattern produced by first microfluidic channels 38 and each line of the second reagent pattern produced by second microfluidic channels 40. However, it is possible to design first and second channel plates 34, 36 with first and second predetermined reagent patterns 38A, 40A having more than one intersection, point between each set of lines. Further, the first and second reagent distribution patterns formed on test chip 32 may in some cases comprise a plurality of discrete reagent spots rather than a continuous line or lines of reagent.

FIG. 10 shows diagrams of first and second channel plates 34, 36 having 384 high density microfluidic channels. FIG. 10A shows a first channel plate 34 having 384 microfluidic channels 38 arranged in a right spiral pattern. The inset shows inlet reservoir 42 for loading a first reagent thereinto. FIG. 10B shows second channel plate 36 having 384 microfluidic channels 40 arranged in a left spiral pattern. The inset shows inlet reservoir 46 for loading a second reagent thereinto. FIG. 10C shows the intersection points of the two spiral patterns of first and second channel plates 34, 36. The intersection points define a dense microarray of 147456 (384*384) test positions 50.

In alternative embodiments, the MMA 30 may be formed from the assembly of one or more additional channel plates. Such additional channel plates may comprise microfluidic channels arranged in a similar pattern to either first or second channel plates 34, 36, or the microfluidic channels may be arranged in other patterns, and may be used to deliver additional reagents, reagent primers or other reagent modifiers, detectors or other materials to test positions 50 on test chip 32.

Various means may be used to induce and regulate the flow of reagent(s) deposited on chip 32 for the purpose of microarray formation and testing. In use, after first channel plate 34 is scaled with test chip 32, one or more first reagents are loaded into inlet reservoirs 42 of first microfluidic channels 38. To initiate the flow of and to distribute the first reagents in first microfluidic channels 38, a force is applied to MMA 30 (FIG. 8E). Various types of forces may be applied to MMA 30 to induce fluid flow, such as centrifugal force applied by spinning MMA 30. The first reagents are then immobilized or fixed on test chip 32. Immobilization of the first reagent may be achieved by various techniques which are known to persons, skilled in the art. For example, immobilization can be achieved by chemical, mechanical, or biochemical methods such as covalent binding, adsorption, cellular adhesion, protein-protein interactions, polymer encapsulation and so forth. As described further below, one example of chemical immobilization is Schiff-base linkage formed between amine and aldehyde groups on test chip 32. The first reagent may comprise probe nucleic acid molecules.

After the first reagent is distributed and immobilized on test chip 32 as described above, first channel plate 34 is then removed. In the next step, second channel plate 36 is sealed with test chip 32. One or more second reagents are loaded into inlet reservoirs 46 of second microfluidic channels 40. A force is applied to MMA 30 (FIG. 8F) to cause the second reagents to flow and become distributed through second microfluidic channels 40. If necessary, a priming reagent or other reagent for modifying or labeling the second reagents may also be applied through second microfluidic channels 40. At test positions 50, the first reagents are exposed to the second reagents. If the first and second reagents are capable of reacting with one another, this results in a positive test reaction at select test positions 52.

In a further step, the positive test reactions between the first and second reagents are detected using methods which are well known in the art. For example, fluorescence labeling, biotin labeling, reflectance measurements, and so forth can be used. In addition, novel detection methods such as surface plasmon resonance may also be used.

Once reagents are loaded into one or more inlet reservoirs 42, 46, various means may be used to induce fluid flow through microfluidic channels 38, 40, including the application of centrifugal, electrokinetic or hydrodynamic forces. The application of centrifugal force, sometimes referred to as “centrifugal pumping”, provides particular advantages. Centrifugal force may be simply applied by spinning MMA 30 in a disc spinner and avoids the need for complicated fluid handling interfaces. As shown in FIG. 8, distribution of reagents by application of centrifugal force is possible for microfluidic channels 38, 40 arranged in either a radial pattern or a spiral pattern. More particularly, when first channel plate 34 having first microfluidic channels 38 arranged in a radial pattern 38A is sealed against test chip 32, direct centrifugal force (F) is used to distribute the first reagent through microfluidic channels 38 by loading MMA 30 in a spinning device and spinning MMA 30 (FIGS. 8C and 8E). When second channel plate 36 having second microfluidic channels 40 arranged in a spiral pattern 40A is sealed against test chip 32 and the resulting MMA 30 is spun in a spinning device, a component of centrifugal force is used to distribute the second reagent through second microfluidic channels 40 (FIGS. 8D and 8F).

When centrifugal force is used, reagents are loaded into inlet reservoirs 42, 46 at locations near the centre of channel plates 34, 36 respectively. To ensure that all the liquids in inlet reservoirs 42, 46 are distributed into first and second microfluidic channels 38, 40 without spillage, and are retained in outlet reservoirs 44, 48 while spinning the chip, inlet and outlet reservoirs 42, 46, 44, 48 may be disposed at an oblique angle (for example, <90 degrees relative to the central axis of the channel plate). In different embodiments, the reservoirs can carry between 0.1 microlitres and 100 microlitres of reagent depending on the size of channel plates 34, 36 and microfluidic channels 38, 40 formed therein. In one embodiment, the microfluidic channels 38, 40 may be on the order of approximately 60 μm wide and approximately 20 μm deep, although many variations are possible. When MMA 30 is spun, the fluid in the inlet reservoirs 42, 46 is driven into first or second microfluidic channels 38, 40. The fluid then moves outwardly along first or second microfluidic channels 38, 40 until it reaches corresponding outlet reservoirs 44, 48 near the periphery of MMA 30, thereby distributing the reagents along the length of microfluidic channels 38, 40.

The flow speeds of the reagents in first or second microfluidic channels 38, 40 can be controlled by adjusting the rotation speed of MMA 30. For example, the flow speeds can be between 200 rpm and 10,000 rpm. Thus, the residence time or the reaction time of reagents can be controlled, i.e. the time can be adjusted to be long enough to allow for reactions, but short enough to save analysis time.

As discussed above, first and/or second microfluidic channels 38, 40 may be arranged in a spiral shape in one embodiment of the invention. It will be appreciated by persons skilled in the art that any type of spiral shape may be used. However, to achieve uniform and quantitative hybridization (or other types of reactions), it is desirable to ensure an approximately constant flow velocity of liquid reagents in the spiral microfluidic channels 38, 40. If the sample volume of the reagents is many times larger than the channel volume, this constant velocity design for spiral microfluidic channels may not be necessary because there is continuous liquid flow in the microfluidic channels. However, when a small volume of reagent is used (e.g. 1 μL), an approximately constant flow velocity of liquid reagents is desirable.

In an example embodiment, MMA 30 is used for testing nucleic acid hybridations, such as DNAs, RNAs, cDNAs or other nucleic acids. For example, the first reagent may comprise DNA probes while the second reagent may comprise samples for testing. In the first step, first channel plate 34 having first microfluidic channels 38 arranged in first predetermined pattern 38A, such as a radial pattern, is sealed with test chip 32, such as an aldehyde glass slide. Next, solutions of aminated DNA probes are loaded into inlet reservoirs 32 and distributed through first microfluidic channels 38 using centrifugal force as described above. The DNA probes become immobilized onto test chip 32 due to Schiff-base linkage formed between amine and aldehyde groups. The DNA probes will form an array on test chip 32 in the same pattern as first predetermined pattern 38A. First channel plate 34 is then removed from test chip 32 and the procedure for reduction of Schiff-base linkages and excess aldehyde moieties is performed. Other methods for immobilizing or fixing the Probes to the test chip 32 can also be used.

In the second step, second channel plate 36 having second microfluidic channels 40 arranged in second predetermined pattern 40A, such as a spiral pattern, is sealed against test chip 32, and samples are introduced into inlet reservoirs 46 and distributed through second microfluidic channels 40 using centrifugal force. As the samples flow through second microfluidic channels 40 of second channel plate 36, the probes are exposed to the samples at test positions 50. Any samples which are complementary to any of the probes become hybridized at select test positions 50, thus indicating a positive test result. In the final step, detection of hybridization of samples on test chip 32, with or without removing second channel plate 36, is then conducted.

In some embodiments, the sample or second reagent comprises complexes 18 comprising nanoparticles 16 that are non-covalently associated with single-stranded nucleic acid molecules 14. Use of nanoparticles (e.g., gold nanoparticles) in hybridization assays have been described in earlier sections of this disclosure. In some embodiments, the use of nanoparticle 16 increases the degree of discrimination between a perfectly matched target sequence and a mismatched sequence in hybridization assays. In some embodiments, the incubation of complexes 18 with probe nucleic acid molecules 10 is performed at ambient temperature or room temperature. For example, the hybridization step may be performed at a temperature between 20 to 30 degrees Celsius, for example, between 22 and 26 degrees Celsius, or between 22 and 24 degrees Celsius, or between 24 and 26 degrees Celsius. Advantageously, the use of an incubation oven that provides elevated temperatures is not required.

To detect hybridized samples on test chip 32, samples could be labeled, and only hybridized samples will remain bound to test chip 32 and be detected. For instance, the sample can be fluorescently labeled in which only the hybridized regions are fluorescent, or the sample can be biotin-labeled in which strept(avidin)-tagged microbeads, after binding, can be detected by reflectance measurement. Alternatively, a detection probe which interacts with hybridized samples only, but not to probes, could be used to detect hybridization. Other methods of detecting hybridized samples are known to persons skilled in the art.

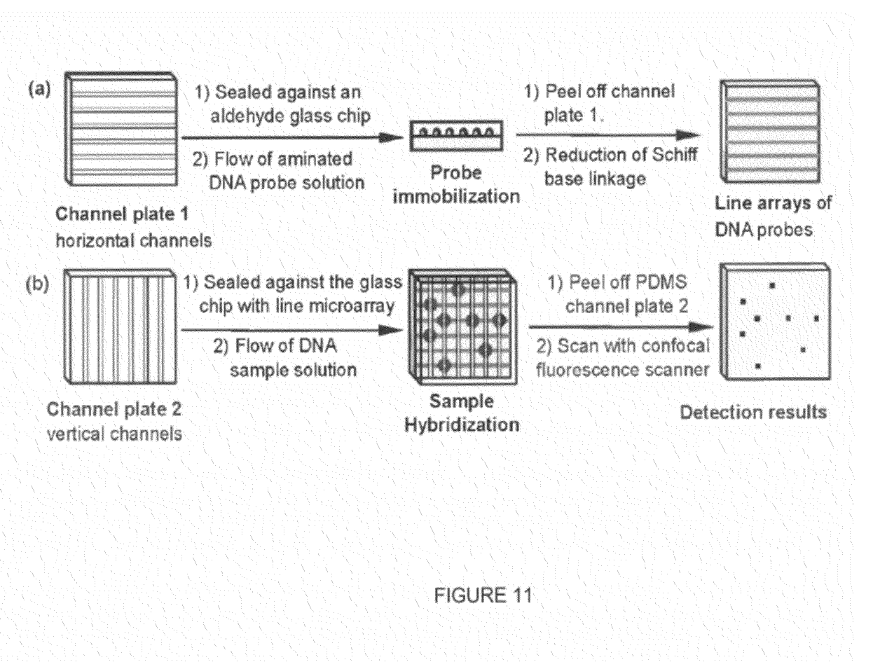

FIG. 11 illustrates another example of MMA comprising parallel straight microchannels. The non-centrifugal MMA method consists of two steps of an assembly process (see FIG. 11). In the first step, channel plate 1 is assembled with the glass chip via reversible bonding. Aminated DNA probes are introduced into the microchannels and are immobilized on the glass chip. A line microarray of probes is thus created. After plate 1 is peeled off, channel plate 2 is then assembled with the same glass chip. The linear microchannels in plate 2 are orthogonal to the linear microchannels in plate 1. The sample solution that flows through the microchannels in plate 2 will intersect the line microarray, and hybridization assays are then performed at the intersections. Typically, a very small sample volume (<1 μL) is need for the hybridization assay using this MMA. The MMA also prevents evaporation and cross-contamination of sample solutions. In some embodiments, the MMA may be PDMS channel plates.

### EXAMPLES

The following examples are provided to illustrate embodiments of the present invention but they are by no means intended to limit its scope.

Surface Modification of Glass Chips. The glass substrates were chemically modified to produce aldehyde-functionalized surfaces using an established procedure (see H. Wang et al., Nucleic Acid Research, 2002, 30, 1-9, which is hereby incorporated by reference). Briefly, plain glass slides were cleaned with a 10% NaOH solution for 10 min at ˜100° C. After being rinsed with distilled water, the slides were treated with a piranha solution (70:30 v/v sulfuric acid to 30% hydrogen peroxide) for 1 h at ˜80° C. The slides were then rinsed with water and dried under a stream of nitrogen. The cleaned slides were treated with a mixture of ethanol/H2O/APTES (95:3:2 by volume) for 2 h under stirring, rinsed with 95% ethanol and deionized H2O, dried under nitrogen, and baked at ˜120° C. for 1 h. The aminated glass slides were then immersed in 5% glutaraldehyde in a 10× phosphate-buffered saline (PBS) solution overnight and washed with acetone and deionized H2O. After being dried in a nitrogen gas stream, the aldehyde-modified glass slides were stored in a dark place at 4° C. before probe printing.

Fabrication of the PDMS-glass microchip. PDMS channel plates consisting of 16 parallel microchannels were fabricated, as follows. A 2 in.×2 in. PDMS channel plate was fabricated using a photolithographic method. The channel pattern was designed using Visual Basic (Microsoft) and was printed on a transparency to create the photomask at a resolution of 3368 dpi. Molding masters were fabricated in a modular clean room (577 series, Clean Air Products, Minneapolis, Minn.). First, a 4 in. silicon wafer was spin-coated with a layer of SU-8 photoresist by a spin coater (WS-400, Laurell Technologies Corp. North Wales, Pa.). Then the channel patterns were created on the SU-8 coated wafer with the photomask using a UV exposure system (model LS-150-3, Bachur & Associates, San Jose, Calif.). The SU-8-coated wafer was developed to produce the molding master. PDMS prepolymer was cast against the molding master and cured at 50° C. for 12 h to yield an elastomeric channel plate. The width of the straight channels was 300 μm, and the channel height was 20 μm. The length of the straight section of each channel was 30 mm. Solution reservoirs (1 mm in diameter) at both ends of channels were punched on the PDMS channel plate using a flat-end syringe needle.

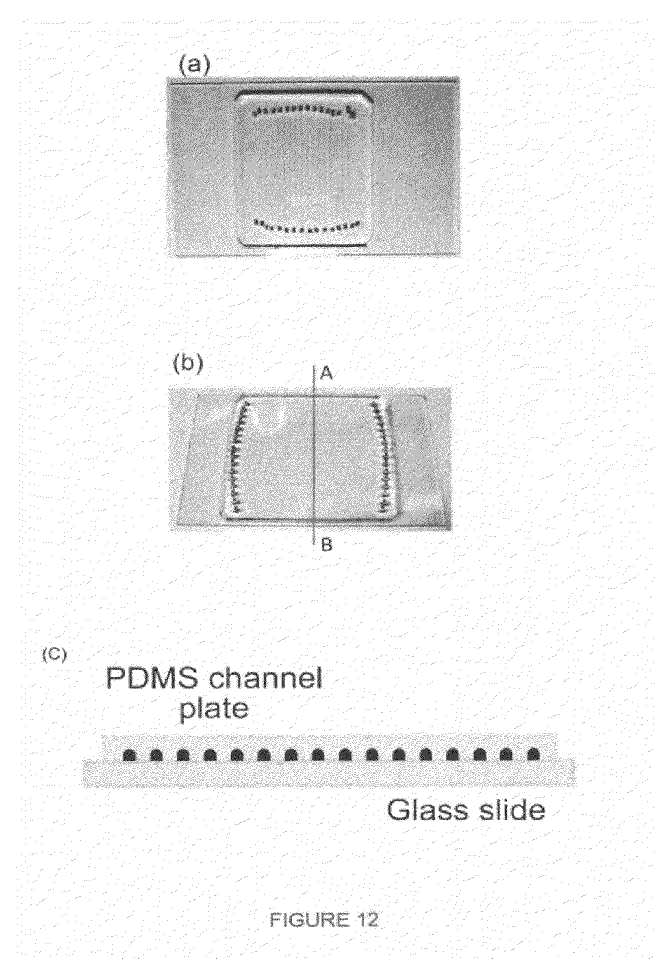

Probe line array creation. The glass slides were chemically modified to produce amine or aldehyde-functionalized surfaces. As shown in FIG. 11 and FIG. 12, the PDMS channel plate was sealed against a glass slide to assemble the microchip. Then, 0.8 μL of probe DNA prepared in the spotting solution (1.0 M NaCl+0.15 M NaHCO3) was added into the inlet reservoirs using a micropipet. The probe solution was filled through the channels by applying vacuum pumping at the outlets. With incubation at room temperature for 30 min, covalent Schiff linkage was formed between the amine ends of the probe oligonucleotides and the aldehyde groups on the glass surface. After the microchannels had been washed with 1 μL of washing solution (0.15% Triton-X 100, 1.0 M NaCl, and 0.15 M NaHCO3), the PDMS channel plate was then peeled off and the glass slide was chemically reduced with a NaBH4 solution (100 mg of NaBH4 dissolved in 30 mL of 1×PBS and 10 mL of 95% EtOH) for 15 min to reduce the Schiff linkage to the more stable C—N single bond. The glass chip was then rinsed with deionized water for 2 min and dried by nitrogen gas and was ready for hybridization. All procedures were conducted at room temperature.

DNA samples. Oligonucleotides were synthesized and modified by Sigma-Genosys Oakville, ON, Canada or International DNA Technologies, Coralville, Iowa. 21-mer DNA probes were modified with an amine group at the 5′-end. Target oligonucleotides are 50-mer with Cy5 dye at the 5′-ends. The central 21 bases are complementary (perfect match) or one base-pair mismatch to the sequences of the probe molecules.

The 21-mer probe sequence is CGCCAGAGAATACCAAAACTC. The sequence of the perfectly matched 50-mer oligonucleotide is CGACATTAA TAAAAAGAGTTTTGGTATTCTCTGGCGAGCATACAAGGCCC. The sequence of the single-base-pair mismatched 50-mer oligonucleotide is CGACATTAATAAAAAGAGTTTTGGTTTTCTCTGGCGAGCATACAAGGC CC.

Two 264 hp PCR products or amplicons were amplified from genomic DNA samples, and were labeled with Cy5 dyes. The central sequences of the sense strand of perfect matched PCR amplicons are complementary to the sequences of probe molecules, while the mismatched amplicons have one base-pair difference from that of the perfect matched ones.

The sequence of the perfectly matched PCR product is:

| | |
|---|---|
| 1 | TTACAGAGTT CATGCCCGAA AGGGTAGACC TCCCACCCTT GTGTATTATT ACTTTGTTGC |
| 61 | TTTGGCGAGC TGCCTTCGGG CCTTGTATGC TCGCCAGAGA ATACCAAAAC TCTTTTTATT |
| 121 | AATGTCGTCT GAGTACTATA TAATAGTTAA AACTTTCAAC AACGGATCTC TTGGTTCTGG |
| 181 | CATCGATGAA GAACGCAGCG AAATGCGATA AGTAATGTGA ATTGCAGAAT TCAGTGAATC |
| 241 | ATCGAATCTT TGAACGCACA |

The sequence of the single-base-pair mismatched PCR product is:

| | |
|---|---|
| 1 | TTACAGAGTT CATGCCCGAA AGGGTAGACC TCCCACCCTT CTGTATTATT ACTTTGTTGC |
| 61 | TTTGGCGAGC TGCCTTCGGG CCTTGTATGC TCGCCAGAGA AAACCAAAAC TCTTTTTATT |
| 121 | AATGTCGTCT GAGTACTATA TAATAGTTAA AACTTTCAAC AACGGATCTC TTGGTTCTGG |
| 181 | CATCGATGAA GAACGCAGCG AAATGCGATA AGTAATGTGA ATTGCAGAAT TCAGTGAATC |
| 241 | ATCGAATCTT TGAACGCACA |

Preparation of DNA-GNP (Gold Nanoparticle) conjugates. Sample DNA molecules were bound to GNPs to form DNA-GNP conjugates before hybridizations. GNP solutions (5 nm in average diameter, Sigma life science) were added into the DNA samples (oligonucleotides or PCR products) in water. In the case of PCR products, the mixtures were incubated at 95° C. to denature and uncoil the DNA chains and so ss-DNA molecules were produced for binding to GNP noncovalently. The DNA-GNP conjugates were snap cooled in an ice-water bath before the hybridization experiments. All the conjugate solutions were diluted to the desired concentration before hybridization.

Sample hybridization and result read-out by fluorescent scanning. For DNA target hybridization, the glass chip with probe line arrays was sealed against the second PDMS channel plate. The straight channels were orthogonal to the printed probe lines on the slide. The DNA samples (oligonucleotides or PCR products) were prepared in the hybridization buffer (1×SSC+0.2% SDS). 1.0 μl DNA targets were added to the inlet reservoirs. Sample solutions in different reservoirs were then pumped into the channels by vacuum suction simultaneously applied at the 16 channel outlets. Hybridizations were achieved at the intersections between complementary DNA targets in solution and probe lines, showing the hybridization patches of 300×300 μm². The microchannels were rinsed immediately with 2 μl hybridization buffer following hybridization.

Following the hybridization and washing procedures, the glass slide was scanned on a confocal laser fluorescent scanner (Typhoon 9410, Molecular Dynamics, Amersham Biosystems) at 25 μm resolution. The excitation and emission wavelengths are 633 and 670 nm. respectively. The photomultiplier tube voltage was set to 600 V. The scanned image was analyzed by IMAGEQUANT 5.2 software. The average fluorescent signals were measured in relative fluorescent unit.

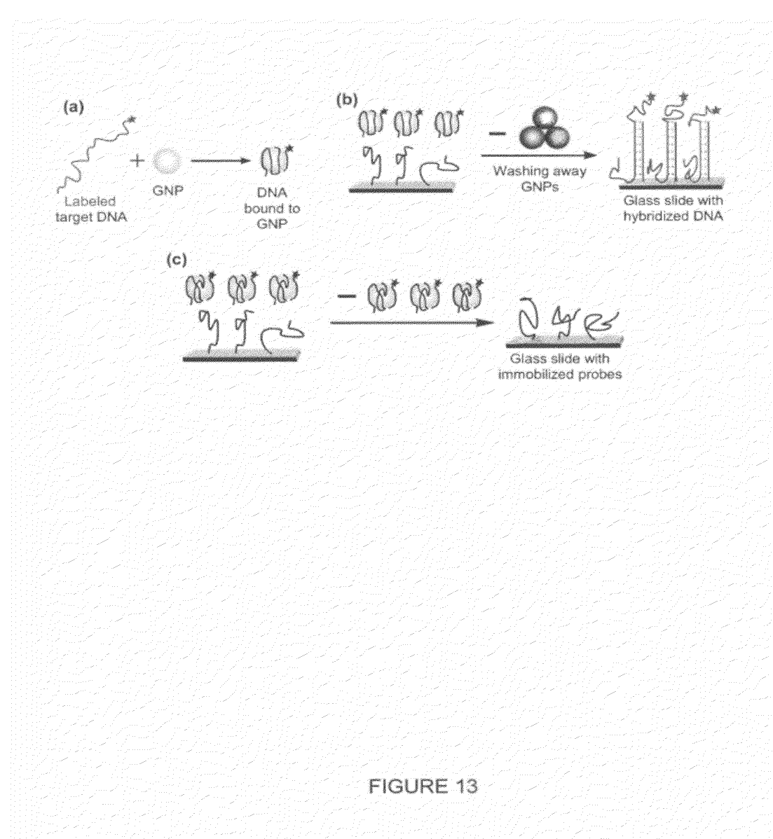

Single base-pair discrimination. The principle of nanoparticle-assisted DNA discrimination was illustrated in FIG. 13. This is based on non-covalent binding between GNPs and targets, in competition with that between targets and immobilized probes. This non-covalent binding is thought to act between GNPs and nitrogen bases on the DNA chains, and can be resulted from both hydrophobic interaction and electrostatic interaction. Here, target DNA labeled with fluorescent molecules were first incubated with GNPs solutions at high temperature in solution. The solution of GNP-DNA conjugates were then applied to DNA probes preprinted microfluidically on the glass slide through the microfluidic method.

Because the base-pair interaction between matched DNA chains is strong, sample DNA molecules could desorb from GNPs and were hybridized with the immobilized probes. The read-out of the hybridization signals were achieved through the fluorescent labels on the sample DNA molecules. On the contrary, mismatched DNA shows much less binding energy with the probes and thus still bind with GNPs. The conjugates would be washed away in the microfluidic flow and discrimination was made.

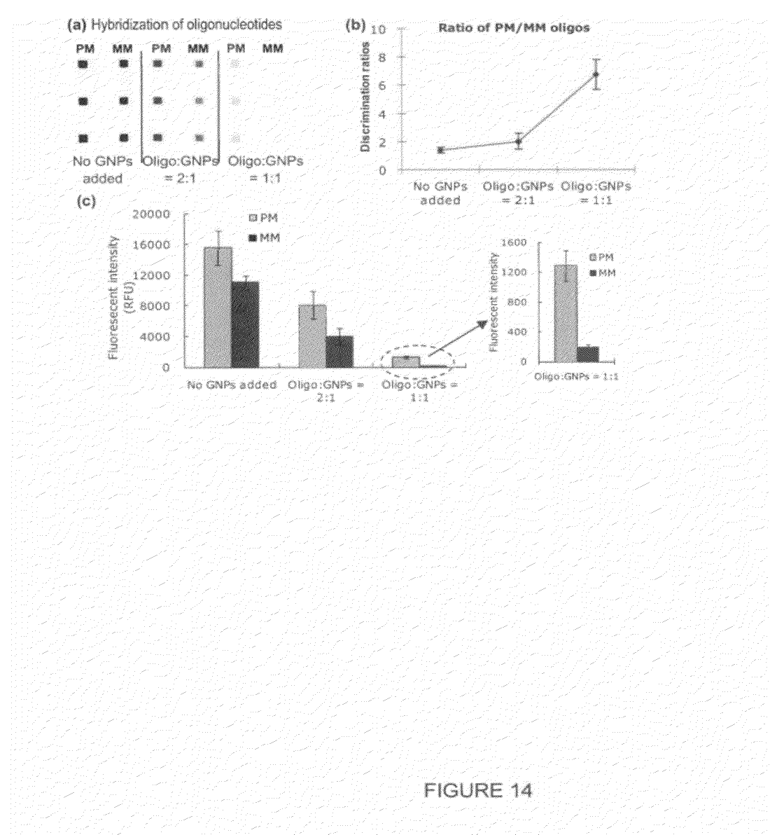

The nanoparticle-assisted discrimination method was shown with two 50-mer oligonucleotides with one-base difference in center. The samples hybridized with the same probe molecules to produce two types of duplexes, namely, the PM duplex and the MM duplex. As shown in the images in FIG. 14(a), without pre-incubation with GNPs, the hybridization signals from mismatched targets are very close to those, obtained from complementary targets, indicating a high degree of non-specific binding between the MM sequence and the probe. The discrimination ratio, which is the hybridization signal ratio of PM duplexes over MM duplexes, is around 1.4. With the use of GNP conjugates instead of free DNA molecules, the discrimination ratio was raised up to 6.8. A clear discrimination between two oligonucleotides was observed from the images in FIG. 14(a).

The effect of the molar ratio between GNPs and target oligonucleotides on hybridization signals and discrimination ratios was also investigated. The molar concentration of GNPs can be calculated from the total gold concentration as well as the size of the GNPs. For 5 nm diameter GNPs in our study, the particle molar concentration is around 86 nM. Conjugates of different GNP/DNA ratios were thus prepared in this manner. FIG. 14(c) compares different hybridization intensities from conjugates of different oligo/GNP ratios (2:1 and 1:1, respectively). It was found that the more GNPs were incubated with DNA samples, the weaker were the hybridization signals. This observation could be explained by the relatively strong binding between GNPs and DNA as well as the slow kinetics of desorption. Despite the reduction in the fluorescent intensities, GNPs do enhance the discrimination of single base-pair mismatch, and the hybridization signals are still adequate as shown in both the images and the inset graph in FIG. 14(c).

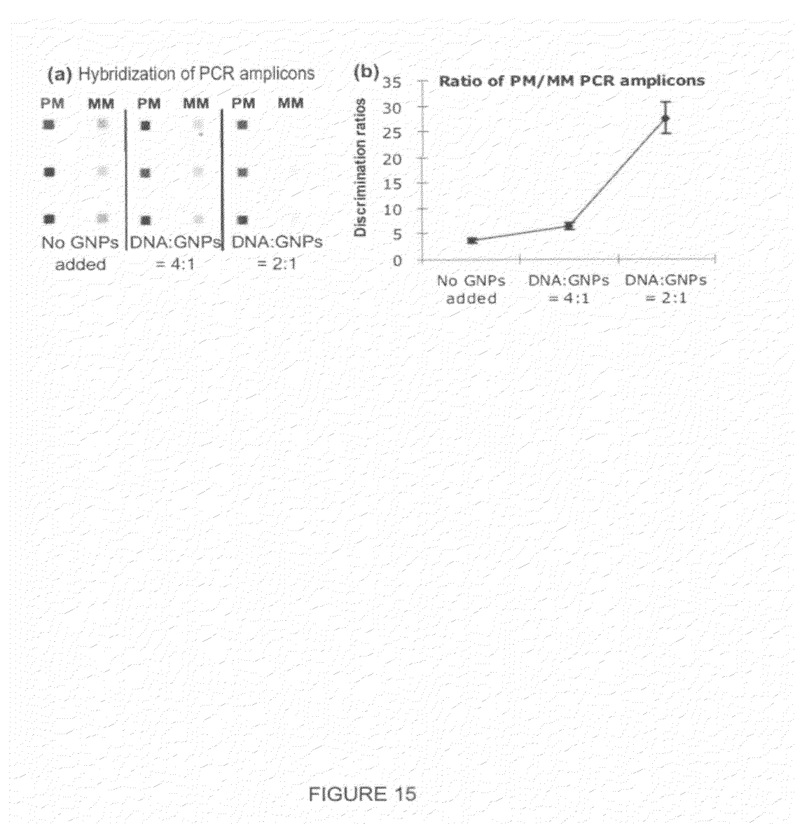

The nanoparticle-assisted microfluidic method was applied to the room-temperature discrimination of two related Botrytis subspecies, B. cinerea and B. squamosa. The two PCR amplicons differ in only one base pair in the middle of the 264 bp long sequence. The amplicons were first incubated at 95° C. with GNPs. This incubation serves for two purposes: one is to denature double-strand amplicons as the usual procedures and another is to promote the subsequent binding of ss-DNA to GNPs. The later snap chilling procedures (at 4° C.) prevented the renaturation of ss-DNA molecules. Although both of the two complementary strands were bound to GNPs and coexisted in the same solutions, they can be still used as samples for later microarray hybridization because the renaturation between long ss-DNA of high complexity is much slower than that between long ss-DNA with short oligonucleotide probes. The discrimination ratio without the use of GNPs is 3.6 at room temperature while it goes up to 27.7 with the assistance of nanoparticles at room temperature. (FIG. 15). Without the use of GNP and using temperature stringency at 50° C., the discrimination ratio is 6.7.

This indicates that the nanoparticle-assisted method has not only improved discrimination, but also alleviated the need of high temperature and related heating devices in the microfluidic chip applications.

As will be apparent to those skilled in the art in the light of the foregoing disclosure, many alterations and modifications are possible in the practice of this invention without departing from the spirit or scope thereof.

What is claimed is:

1. A nucleic acid hybridization method, comprising:
(a) providing complexes comprising nanoparticles non-covalently associated with target nucleic acid molecules;
(b) providing probe nucleic acid molecules immobilized on a surface;
(c) incubating said complexes with said immobilized probe nucleic acid molecules; and
(d) detecting the presence of duplexes on said surface each comprising a strand of one of said target nucleic acid molecules and a strand of one of said probe nucleic acid molecules.

2. A method according to claim 1, wherein said complexes are provided by mixing said nanoparticles and said target nucleic acid molecules.

3. A method according to claim 2, comprising denaturing said target nucleic acid molecules prior to step (a).

4. A method according to claim 3, wherein said denaturing is performed at a temperature between 85° C. and 100° C.

5. A method according to claim 4, comprising cooling said target nucleic acid molecules to 4° C. immediately after said denaturing step.

6. A method according to claim 1, comprising washing said surface before said detecting.

7. A method according to claim 1, wherein said incubating is performed at ambient temperature.

8. A method according to claim 1, wherein said incubating is performed at a temperature below 30° C.

9. A method according to claim 1, wherein said incubating is performed at a temperature between 20° C. and 25° C.

10. A method according to claim 1, wherein said nanoparticles comprise gold nanoparticles.

11. A method according to claim 1, wherein said nanoparticles comprise silver nanoparticles.

12. A method according to claim 1, wherein said nanoparticles comprise metal nanoparticles.

13. A method according to claim 1, wherein said nanoparticles comprise semi-conductor nanoparticles.

14. A method according to claim 1, wherein said nanoparticles comprise non-metal nanoparticles.

15. A method according to claim 1, wherein said nanoparticles are magnetic or magnetically attractable.

16. A method according to claim 1, wherein said nanoparticles are coated with negative charged ions.

17. A method according to claim 1, wherein said nanoparticles are coated with citrate.

18. A method according to claim 1, wherein said nanoparticles are generally spherical.

19. A method according to claim 1, wherein said nanoparticles are generally rod-shaped.

20. A method according to claim 1, wherein said nanoparticles are sized between 1 to 100 nanometers.

21. A method according to claim 1, wherein said nanoparticles are sized between 3.5 to 6.5 nanometers.

22. A method according to claim 1, wherein said nanoparticles have an average size of 5.0 nanometers.

23. A method according to claim 1, wherein said target nucleic acid molecules comprise DNA.

24. A method according to claim 1, wherein said target nucleic acid molecules are derived from double-stranded DNA.

25. A method according to claim 1, wherein said target nucleic acid molecules comprise single-stranded DNA.

26. A method according to claim 1, wherein said target nucleic acid molecules comprise RNA.

27. A method according to claim 1, wherein said target nucleic acid molecules comprise oligonucleotides.

28. A method according to claim 1, wherein said target nucleic acid molecules are labeled with a detectable label.

29. A method according to claim 1, wherein said target nucleic acid molecules are labeled with a fluorescent label.

30. A method according to claim 1, wherein said probe nucleic acid molecules comprise DNA.

31. A method according to claim 1, wherein said probe nucleic acid molecules comprise double-stranded DNA.

32. A method according to claim 1, wherein said probe nucleic acid molecules comprise single-stranded DNA.

33. A method according to claim 1, wherein said probe nucleic acid molecules comprise RNA.

34. A method according to claim 1, wherein said probe nucleic acid molecules comprise oligonucleotides.

35. A method according to claim 1, wherein said probe nucleic acid molecules comprise a chromosome preparation suitable for fluorescence in situ hybridization (FISH).

36. A method according to claim 1, wherein said probe nucleic acid molecules are labeled with a detectable label.

37. A method according to claim 1, wherein said probe nucleic acid molecules are labeled with a fluorescent label.

38. A method according to claim 1, wherein said surface comprises a generally flat surface.

39. A method according to claim 1, wherein said surface comprises a curved surface.

40. A method according to claim 1, wherein said surface is the surface of a bead.

41. A method according to claim 1, wherein said surface is formed from a material selected from the group consisting of glass, silicone, plastic, polymer and cellulose.

42. A method for distinguishing two target nucleic acid molecules whose nucleotide sequences differ by at least one nucleotide, the method comprising:
carrying out two separate nucleic acid hybridization assays in parallel, the first assay with a first target and a probe, the second assay with a second target and the same probe, each assay comprising:
a) mixing a target nucleic acid with nanoparticles in a sample solution to form complexes comprising the nanoparticles non-covalently associated with the target nucleic acid molecules:
b) incubating said sample solution with probe nucleic acid molecules immobilized on a surface; and
c) detecting the presence of target:probe duplex on the surface;
whereby the two target nucleic acid molecules are distinguished by different degrees of hybridization to the probe.

43. A method for distinguishing two target nucleic acid molecules whose nucleotide sequences differ by at least one nucleotide, the method comprising:
a) label the first target nucleic acid with a first detectable label and label the second target nucleic acid with a second detectable label;
b) combine the first target nucleic acid and the second nucleic acid;
c) mixing the first and second target nucleic acid molecules with nanoparticles in a sample solution to form complexes comprising the nanoparticles non-covalently associated with the target nucleic acid molecules;
d) incubating said sample solution with probe nucleic acid molecules immobilized on a surface; and
e) detecting the presence of target:probe duplex on the surface;
whereby the two target nucleic acid molecules are distinguished by different degrees of hybridization to the probe.

44. A microarray method comprising:
a) providing a solid support,
b) immobilizing a plurality of nucleic acid probes at discrete positions on the support,
c) incubating a sample solution with the probes, the sample solution comprising nanoparticles non-covalently associated with sample nucleic acid molecules,
d) determining the degree of hybridization between the sample and the probes.

45. A method according to claim 44, wherein said incubating comprises incubating at a temperature below 30 degrees Celsius.

46. A method according to claim 44, wherein said incubating comprising incubating at a temperature between 22 and 26 degrees Celsius.

47. A method according to claim 44, wherein the sample nucleic acid molecules are labeled with a fluorophore.

48. A method according to claim 44, wherein the sample nucleic acid molecules comprises first sample nucleic acid molecules obtained from a first sample source and second sample nucleic acid molecules obtained from a second sample source.

49. A method according to claim 48, wherein the first sample nucleic acid molecules and the second sample nucleic acid molecules are labeled with two different fluorophores.

50. A method according to claim 48, wherein the first sample nucleic acid molecules and the second sample nucleic acid molecules comprise nucleotide sequences that differ by two nucleotides.

51. A method according to claim 48, wherein the first sample nucleic acid molecules and the second sample nucleic acid molecules comprise nucleotide sequences that differ by a single nucleotide.

52. A method of forming a microfluidic microarray assembly (MMA) comprising: (a) providing a test chip; (b) providing a first channel plate scalingly connectable to said test chip for applying at least one first reagent to said test chip, wherein said first channel plate comprises a plurality of first microfluidic channels configured in a first predetermined reagent pattern; (c) assembling said first channel plate to said test chip; (d) flowing said at least one first reagent through said first microfluidic channels to form a first array of said at least one first reagent on said test chip in said first predetermined reagent pattern; (e) immobilizing said at least one first reagent on said test chip at least some test locations of said first array; (f) removing said first channel plate from said test chip; (g) providing a second channel plate sealingly connectable to said test chip for applying at least one second reagent to said test chip, wherein said second channel plate comprises a plurality of second microfluidic channels configured in a second predetermined pattern differing from said first predetermined pattern; (h) assembling said second channel plate to said test chip; and (i) flowing said at least one second reagent through said second microfluidic channels to form a second array of said at least one second reagent on said test chip in said second predetermined reagent pattern, wherein said second array intersects said first array at said test locations, and wherein the second reagent comprises complexes comprising nanoparticles non-covalently associated with sample nucleic acid molecules.

53. A microarray device comprising:
a) a test chip comprising a plurality of discrete, spatially predetermined test positions, each of the test positions being located at the intersection between a first predetermined reagent pattern and second predetermined reagent pattern;
b) at least one first reagent immobilized on said test chip at said test positions; and
c) a channel plate sealingly connected to said test chip, said channel plate comprising a plurality of microfluidic channels for distributing at least one second reagent on said test chip in said second predetermined reagent pattern, said second reagent comprising nanoparticles non-covalently associated with sample nucleic acid molecules.


SEQUENCE LISTING (5 sequences)

SEQ ID NO: 1 - DNA, 21 bp, Artificial Sequence
  Synthetic 21-mer probe
cgccagagaataccaaaactc

SEQ ID NO: 2 - DNA, 50 bp, Artificial Sequence
  Synthetic 50-mer oligonucleotide perfectly matched to SEQ ID NO1
cgacattaataaaaagagttttggtattctctggcgagcatacaaggccc

SEQ ID NO: 3 - DNA, 50 bp, Artificial Sequence
  Synthetic 50-mer oligonucleotide single-base- pair mismatched to SEQ ID NO1
cgacattaataaaaagagttttggttttctctggcgagcatacaaggccc

SEQ ID NO: 4 - DNA, 260 bp, Artificial Sequence
  Synthetic PCR product of SEQ ID NO2
ttacagagttcatgcccgaaagggtagaccgcccacccttgtgtattattactttgttgctttggcgagctgccttcgggccttgtatgctcgccagagaataccaaaactctttttattaatgtcgtctgagtactatataatagttaaaactttcaacaacggatctcttggttctggcatcgatgaagaacgcagcgaaatgcgataagtaatgtgaattgcagaattcagtgaatcatcgaatctttgaacgcaca

SEQ ID NO: 5 - DNA, 260 bp, Artificial Sequence
  Synthetic PCR product of SEQ ID NO3
ttacagagttcatgcccgaaagggtagaccgcccacccttgtgtattattactttgttgctttggcgagctgccttcgggccttgtatgctcgccagagaaaaccaaaactctttttattaatgtcgtctgagtactatataatagttaaaactttcaacaacggatctcttggttctggcatcgatgaagaacgcagcgaaatgcgataagtaatgtgaattgcagaattcagtgaatcatcgaatctttgaacgcaca